(19) United States
(12) Patent Application Publication
(10) Pub. No.: US 2026/0047818 A1
(43) Pub. Date: Feb. 19, 2026

(54) LEAKY JOINT SYNDROME DIAGNOSTICS AND TREATMENTS

(71) Applicant: Pawel G. Ochalski, York, PA (US)

(72) Inventor: Pawel G. Ochalski, York, PA (US)

(21) Appl. No.: 19/101,048

(22) PCT Filed: Aug. 4, 2023
(86) PCT No.: PCT/US2023/029546

§ 371 Date: Feb. 4, 2025

Related U.S. Application Data
(60) Provisional application No. 63/370,548, filed on Aug. 5, 2022.
(60) Provisional application No. 63/370,549, filed on Aug. 5, 2022.

(51) Int. Cl. A61B 8/08; A61K 51/10; G01N 33/53

(52) U.S. Cl. CPC A61B 8/0875; A61K 51/1018; G01N 33/5308; A61K 2123/00; G01N 2400/40; G01N 2800/10

(57) ABSTRACT

Disclosed are methods for detecting extracapsular synovial fluid or components thereof as a marker for initiation of processes that lead to degenerative joint disease in a subject.

This application claims priority from U.S. Provisional Application No. 63/370,549 filed on Aug. 5, 2022 and U.S. Provisional Application No. 63/370,548 filed on Aug. 5, 2022, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### FIELD OF THE INVENTION

The present invention relates to a method of detection of biomarkers associated with leaky back syndrome and diagnostics and treatments regarding the same.

### BACKGROUND OF THE INVENTION

Chronic low back pain is a widespread problem affecting millions of adults in the United States resulting in significant impairment of quality of life and lost productivity. While inflammation is generally accepted as contributing to the symptoms of both acute and chronic episodes of low back pain, identification of an upstream biomarker that activates the secondary inflammatory response has been elusive, thus limiting the development of effective diagnostics and therapeutics. This has been challenging in the initial phases of recurrent episodes of back pain when there is no evidence of radiographic degenerative changes.

### SUMMARY OF THE INVENTION

Methods are disclosed herein for diagnosing early stages of degenerative joint disease, including degenerative lumbar disease. The methods involve detecting synovial fluid outside of a synovial joint (i.e., extracapsular synovial fluid) in an animal, including humans. Generally, the synovial fluid is detected in proximity to the synovial joint from which the synovial fluid originates. The synovial joint can be in the spine, elbow, thumb, foot, wrist, hip, shoulder, knee, and the like. In some examples, the synovial joint includes a facet joint. The facet joint can be a lumbar facet joint. The synovial fluid can leak from the synovial joint. The leakage can be from acute joint effusion. The leakage can be from physiologic gaping, from opening of the synovial joint during endpoints of flexion or extension, and the like. The synovial joint from which the fluid leaks can be strained or injured. The extracapsular synovial fluid can be detected by imaging methods, including computed tomography, magnetic resonance imaging (MRI) or ultrasound. The extracapsular synovial fluid can be detected using, for example, an antibody that binds to a component of the leaked synovial fluid. The antibody can have an attached detectable moiety. The methods can detect components of synovial fluid. A component of synovial fluid can be hyaluronic acid (hyaluronan) and/or fragments thereof.

A method for detecting synovial fluid and/or components of synovial fluid surrounding a synovial joint can include administering to a patient, a substance (e.g., antibody) that can bind a component of extracapsular synovial fluid, and detecting the substance bound to the component of the extracapsular synovial fluid in the body of the patient.

Also disclosed are methods for identifying an individual at risk for developing degenerative lumbar disease including, detecting extracapsular synovial fluid and/or components thereof in proximity to a lumbar facet joint, detecting inflamed tissue surrounding a synovial joint, detecting scarring, scabbing and/or plugs in a lumbar facet joint, or detecting inflammatory molecules in synovial fluid, a tissue sample, or blood.

Generally, the methods for detecting the extracapsular synovial fluid and/or components thereof are used early on in the course of degenerative lumbar disease. Generally, the methods are used when a patient presents with acute back pain, has no radiologic evidence of degenerative back disease and has relatively preserved intervertebral disc. The patient generally does not yet have symptoms of chronic back pain as would be apparent in advanced end-stage degenerative disease. However, the patient can have significant impairment of quality of life with symptoms typically described as morning stiffness, muscle spasms and can have some generalized referred pain into the proximal legs. The patient generally does not have fatty infiltration of the multifidus muscle, nor a synovial cyst. It is the synovial fluid leakage from the lumbar facet joints, which can be recurrent, that initiates inflammation in surrounding tissue and begins the processes that leads to degenerative lumbar disease.

Other objects and advantages of this invention will become readily apparent from the ensuing description.

### BRIEF DESCRIPTION OF THE FIGURES

FIG. 1A shows a schematic of back pain acuity and disease progression.

FIG. 1B shows a continuum of leaky back syndrome and overlap transition into mechanical instability.

FIG. 2A shows a schematic of a non-limiting example of, without wishing to be bound by theory, strain and mechanical response leading to synovial fluid leakage from facet joint leading to inflammatory response.

FIG. 2B shows an example of leaky back syndrome. Initial injury from excessive axial load, rotational force or repetitive task, can in lumbar facet capsular injury with synovial fluid leakage into the para-facet structures. This can result in activation of symptoms of inflammatory back pain and radiographic changes of fat replacement as a result of ongoing episodes of leaky back syndrome.

FIG. 3A shows an example intra-operative image while a patient was undergoing a lumbar fusion for an adjacent level instability. Posterior midline open incision with visualization of the L3-4 and L4-5 posterior elements is shown. For orientation, the patient is lying supine with the head to the left of the image and feet to the right as seen in the inset in the upper left. While we were assessing the spine for abnormal motion using gentle flexion via upward motion of the L3 spinous process (1) we placed the spine in gentle flexion (2) across the L3-4 segment and visualized leakage of synovial fluid from the L3-4 facet capsule on the left (3) as highlighted in yellow. This was reproducible and is the result of facet capsule incompetence.

FIG. 3B shows an example of the logic behind leaky back syndrome.

FIG. 4A-C shows non-limiting MRI images. Sagittal T2 MRI showing overall alignment and level of degenerative changes in a patient with mid-stage leaky back syndrome. (Panel A) note the presence of fatty infiltration of the multifidus muscle and mildly thickened ligament flavum at the L3 level (Panel B) compared to the adjacent level control demonstrating overall less fatty infiltration and ligamentum flavum hypertrophy (Panel C). Higher magnification (inset, Panel B) demonstrates presence of T2 signal within the facet joint and arrows indicate leakage of synovial fluid into the underlying ligaments flavum and surrounding paraspinal musculature, with the latter having been observed intra-operatively.

FIG. 5A-D shows non-limiting MRI images. Axial T2 MRI showing the fatty infiltration of multifidus muscle (Panel A) and mildly thickened ligament flavum compared to a normal adjacent level control (Panel B) in a patient with suspected late-stage leaky facet syndrome. Note the edema within the multifidus muscle (arrow) associated with the loss of the muscle striations along with the fatty infiltration (double arrowhead) which is characteristic of a secondary chronic inflammatory response. Inset shows higher magnification of these changes compared to a normal adjacent level normal control where the muscle cyto-architecture is preserved due to in part from the lower junta-facet inflammatory changes. Axial T2 MRI showing the fatty infiltration of multifidus muscle (Panel C) and mildly thickened ligament flavum compared to a normal adjacent control (Pane D) in a patient with suspected leaky facet syndrome. Note the edema within the multifidus muscle (arrow) associated with the loss of the muscle striations along with the fatty infiltration (double arrowhead) characteristic of a secondary chronic inflammatory response. Inset shows higher magnification of these changes compared to a normal adjacent level normal control where the muscle cyto-architecture is preserved due to in part from the lower junta-facet inflammatory changes.

FIG. 6 shows, without wishing to be bound by theory, embodiments of the disclosure.

FIG. 7 shows a schematic of a radiopharmaceutical tag to localize an extra-capsular synovial fluid leak.

FIG. 8A shows a schematic adapted from (Tisher et al., Eur Spine J (2006) 15: 308-315). The schematic drawing shows the contact points between superior and inferior facets during flexion/extension movements: during flexion, the inferior facet slides over the superior facet and maximal pressure occurs at the superior pole of the superior facet. During maximal extension, the inferior facet comes in contact with the arch of the superior facet.

FIG. 8B shows effects of facet capsule failure and leakage of synovial fluid.

FIG. 9 shows common imaging procedures.

FIG. 10 shows illustrations of facet joints.

FIG. 11 shows an illustration of the anatomy of the spinal column and examples of synovial fluid leakage (O'Leary, Annu. Rev. Biomed. Eng. 2018. 20: 145-70).

FIG. 12 shows non-limiting examples (Biprojit Nath, Introduction to Radiopharmaceuticals Basics, Ideal Radiopharmaceutical, youtu.be/l2TQHPkCXKE) of imaging devices which can be used to diagnose leaky back syndrome. Without wishing to be bound by theory, these devices can be used in tandem with the diagnostics of the disclosure. For example, the diagnostic is a radiopharmaceutical.

FIG. 13 shows a nonlimiting, exemplary schematic depicting the molecular weight and roles of hyaluronan (adapted from www.glycoforum.gr.jp/article/09A1.html).

FIG. 14 shows a non-limiting, exemplary schematic of the degradation of HA (adapted from Krupkova et al., Eur. Spine J. 2020 March; 29(3):605-615).

FIG. 15 shows example scenarios of treatment and diagnosis of back pain. In one embodiment, the methods for diagnosis disclosed herein can result in early diagnosis.

### DETAILED DESCRIPTION OF THE INVENTION

Aspects described herein provide methods and diagnostics for diagnosing and treating a leakage of synovial fluid from synovial joints, the fluid leakage initiating and leading to degenerative back disease. In some embodiments, the synovial fluid leakage from the facet joints can be referred to as Leaky Back Syndrome (LBS).

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “substantially the same” or “substantially” can refer to variability typical for a particular method is taken into account.

The terms “sufficient” and “effective”, as used interchangeably herein, can refer to an amount (e.g., mass, volume, dosage, concentration, and/or time period) needed to achieve one or more desired result(s).

The term “administration” can refer to introducing a composition of the present disclosure into a subject. For example, one route of administration of the composition can be administered by intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, “treat”, “treatment”, and/or “treating” can refer to acting upon a condition (e.g., leaky back syndrome), a disease or a disorder with a composition to affect the condition (e.g., leaky back syndrome), disease or disorder by improving or altering it. The improvement or alteration can include an improvement in symptoms or an alteration in the physiologic pathways associated with the condition (e.g., leaky back syndrome), disease, or disorder. “Treatment” can refer to one or more treatments of the disease or condition in a subject (e.g., a mammal, typically a human or non-human animal of veterinary interest), and can include: (a) reducing the risk of occurrence in a subject determined to be predisposed to the condition or disease but not yet diagnosed with it (b) impeding the development of the condition or disease, and/or (c) relieving the condition or disease, e.g., causing regression of the condition or disease and/or relieving one or more condition or disease symptoms. As used herein, the terms “prophylactically treat” or “prophylactically treating” can refer to completely or partially preventing (e.g., about 50% or more, about 60% or more, about 70% or more, about 80% or more, about 90% or more, about 95% or more, or about 99% or more) a condition (e.g., condition or disease), a disease, or a symptom thereof and/or can be therapeutic in terms of a partial or complete cure for a condition (e.g., condition or disease), a disease, and/or adverse effect attributable to the disease.

As used herein, “therapeutic” can refer to curing or treating a symptom of a disease or condition. For example, the disease or condition can comprise leaky back syndrome.

As used herein, the term “subject,” or “patient,” can include humans and mammals (e.g., mice, rats, pigs, cats, dogs, and horses), and non-mammals (e.g., aves such as chickens etc.). Typical subjects to which compounds of the present disclosure can be administered will be mammals, particularly primates, especially humans. For veterinary applications, a wide variety of subjects will be suitable, non-limiting examples of which comprise livestock such as cattle, sheep, goats, cows, swine; poultry such as chickens, ducks, geese, turkeys; and domesticated animals particularly pets such as dogs and cats. For diagnostic or research applications, a wide variety of mammals can be suitable subjects, non-limiting examples of which comprise rodents (e.g., mice, rats, hamsters), rabbits, primates, and swine such as inbred pigs and the like. The term “living subject” can refer to a subject noted above or another organism that is alive.

As used herein, the term “fluid sample” can refer to a body fluid sample including, without limitation synovial fluid, blood, plasma, cerebrospinal fluid, and other body fluids. The fluid sample can comprise a serologic sample. The body fluid sample can be diluted with, e.g., buffer or other reagents that facilitate handling.

Before explaining at least one embodiment of the disclosure in detail, it is to be understood that the disclosure is not necessarily limited in its application to the details set forth in the following description or exemplified by the examples. The disclosure is capable of other embodiments or of being practiced or carried out in various ways. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Leaky Back Syndrome

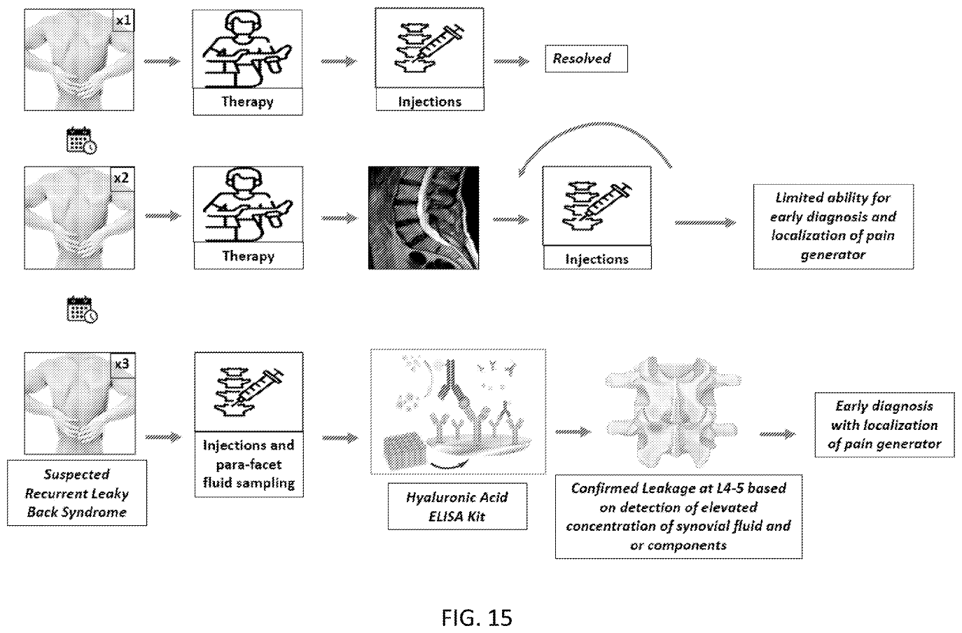

Described herein are diagnostics and methods for diagnosing early stages of degenerative joint disease (FIG. 15). Described herein are diagnostics and treatments for leaky back syndrome (LBS). As used herein, the term “leaky back syndrome” can refer to a pathophysiological process of synovial fluid leakage from synovial joints. As used herein, the term “leaky back syndrome” and the term “leaky joint syndrome” can be used interchangeably. In embodiments, the synovial fluid leakage is from the lumbar facet joints. Without wishing to be bound by theory, the synovial fluid leakage and/or components thereof can activate downstream inflammatory responses resulting in clinical and radiographic manifestations.

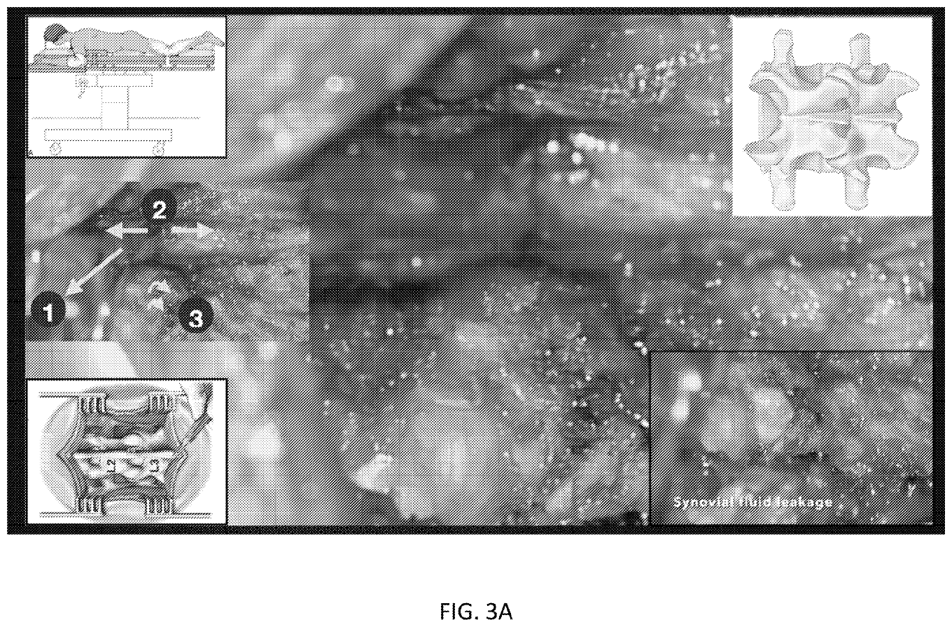

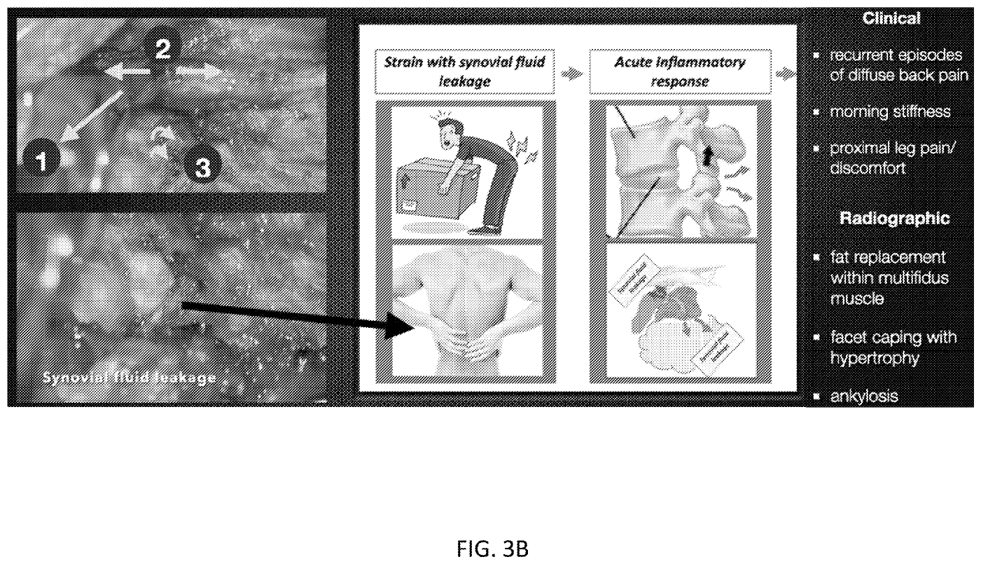

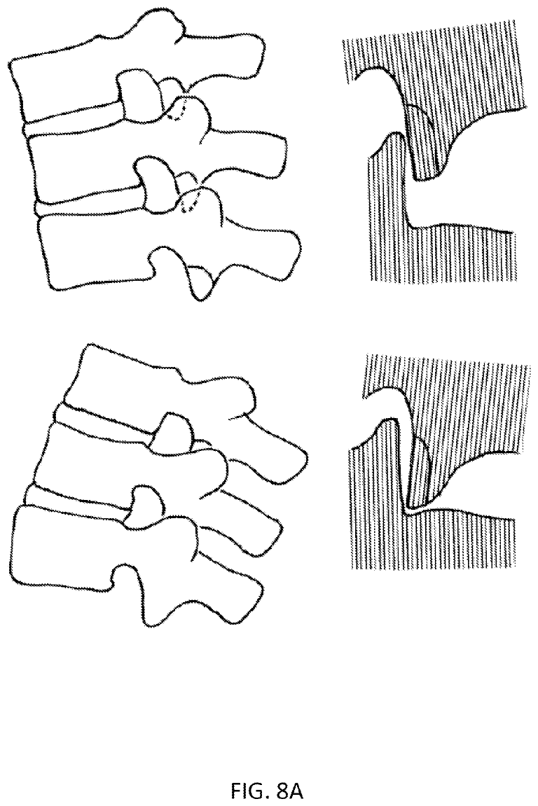

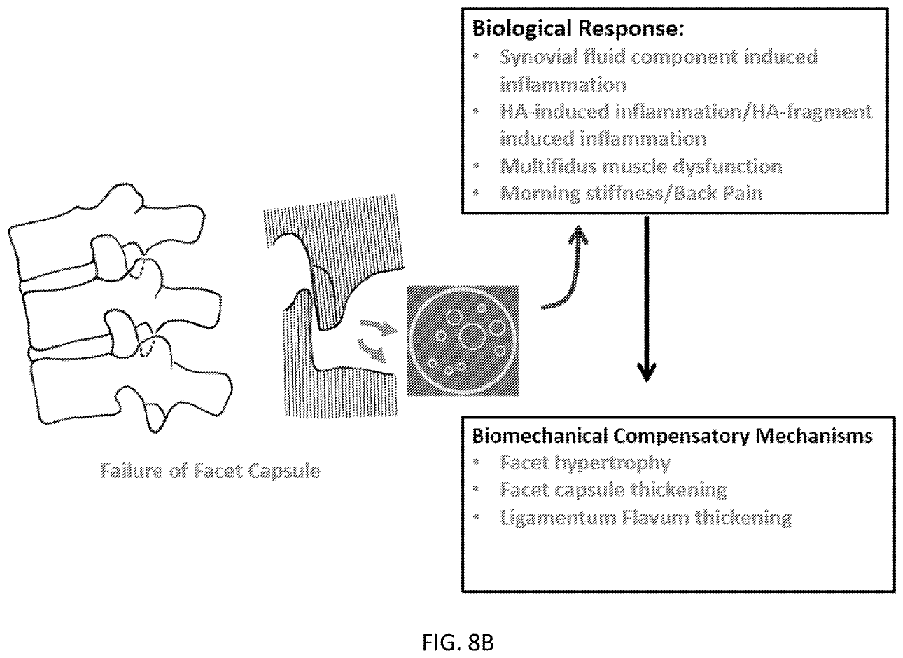

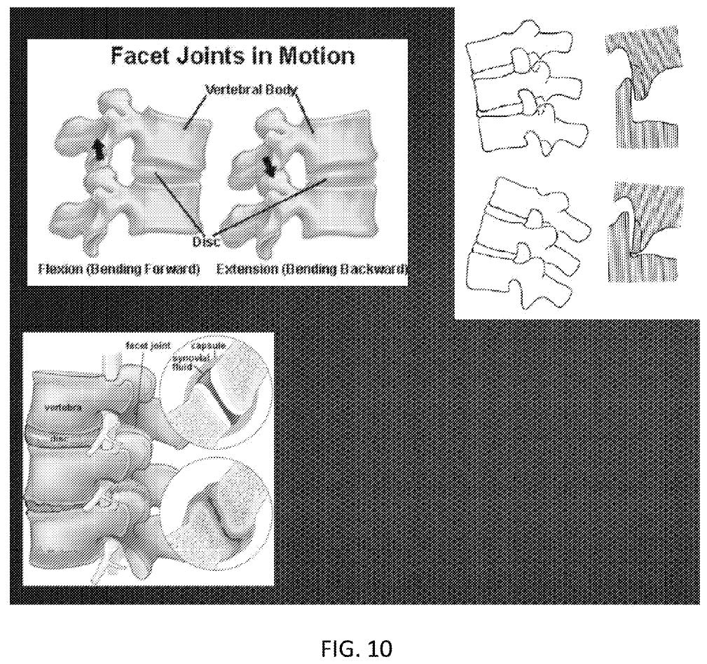

In embodiments, the synovial fluid leaks from the zygapophyseal joints of the spine into the surrounding paraspinal musculature and tissues. This has been observed during back surgeries (FIGS. 3A and 3B). As used herein, zygapophyseal joints can refer to synovial joints in the spine. The joint is composed of two halves of half moon-shaped bones with an intervening synovial membrane (FIGS. 8A and 8B, 10). This membrane produces synovial fluid with visco-elastic properties enhancing axial load sharing during weight bearing activities. The joint and synovial fluid is bound by fibro-cartilaginous capsule separating the joint space from the surrounding tissues.

### Synovial Fluid as a Biomarker for Back Pain and Clinical Classifications Thereof

As discussed herein, various embodiments of the disclosure permit the detection of biomarkers in a sample as an indicator of a particular disease, infection, or condition. In embodiments, biomarkers include any biological marker whose presence or absence is known to be associated with a particular disease, infection, or condition. Biomarkers can be useful for diagnosing a patient with a particular disease, infection, or condition; predicting clinical outcomes of a patient suffering from a disease, infection, or condition, directing individualized treatment decisions for a patient suffering from a particular disease, infection, or condition, predicting the likelihood that a particular treatment will be effective, assessing the effectiveness of a particular treatment response, or a combination thereof. Biomarkers can be specific for a particular disease, infection, or condition, showing little to no cross-over to alternate disease states.

Aspects of the invention are drawn towards a method of detecting synovial fluid and/or components thereof outside of a synovial joint as a biomarker for back pain and early stage of degenerative joint disease. For example, the synovial fluid and/or components can be detected visually as a biomarker. Aspects of the invention are drawn towards a biomarker for early degenerative disease in a subject. For example, the degenerative disease is a degenerative lumbar disease. In embodiments, the method comprises detecting inflammatory molecules, or elevated levels thereof, in a tissue sample obtained from tissue in proximity to a lumbar synovial joint. As used herein, the term “synovial fluid” can refer to the fluid in synovial joints (i.e., in the synovial capsule of a synovial joint). For example, synovial fluid can refer to a thin, lubricating substance within the synovial cavity that reduces friction within the joint. In embodiments, “synovial joint” and “diarthrosis” can be used interchangeably. As used herein, a synovial joint can refer to a joint that joins bones or cartilage with a fibrous joint capsule of synovial capsule that contains synovial fluid. In embodiments, synovial joints can secrete synovial fluid.

Herein, “synovium” can refer to an inner lining of a synovial joint. Joints that are not synovial joints can be fibrous joints or cartilaginous joints. Synovial joints can be pivot joints (e.g., spine), hinge joints (e.g., elbow), saddle joints (e.g., articulation between trapezium carpal bone and first metacarpal bone at base of human thumb), plane joints (e.g., between tarsal bones of foot), condyloid joints (e.g., radiocarpal joint of wrist) or ball-and-socket joints (e.g., hip, shoulder).

Herein, “synovial capsule” can refer to a fibrous capsule of a synovial joint, continuous with the periosteum of the articulating bones, that surrounds the joint capsule and articulating bones. Joint capsules can have an outer fibrous layer and an inner synovial membrane. In embodiments, leaky back syndrome can be caused by a herniated synovial capsule.

Herein, “synovial cyst” can refer to a cyst, generally benign, in or on the synovial joint capsule. Synovial cysts can release synovial fluid. In embodiments, a synovial cyst can be an indication of late-stage LBS.

Herein, “facet joint” can refer to synovial joints in the back/spine that connect the vertebrae in humans. The spine can have cervical (neck), thoracic (midback) and lumbar (low back) facet joints. Facet joints can be referred to as zygapophyseal or a prohyseal joints. Facet joints are the only synovial joints in the spine in humans. The back also contains intervertebral discs, which are not synovial joints. Intervertebral discs contain a protein-based fluid/elastic core with a gelatin-like consistency called the nucleus pulposus. Intervertebral discs can be called annular discs. Two facet joints and one intervertebral disc form a tripod relationship between each vertebra.

In embodiments, the biomarker can comprise extracapsular synovial fluid. In embodiments, the biomarker can comprise one or more components of synovial fluid (e.g., hyaluronic acid and/or fragments thereof). In some embodiments, hyaluronic acid and/or fragments thereof are not components of synovial fluid that are detected in the methods disclosed herein. As used herein, extracapsular synovial fluid can refer to synovial fluid found outside of the joint capsule. In embodiments, the synovial fluid outside of the synovial joint is detected in proximity to a synovial joint. In embodiments, the synovial fluid outside of the synovial joint is detected in proximity to a synovial joint from which the synovial fluid originates.

In some embodiments, the subject does not have a synovial cyst associated with the synovial joint from which the synovial fluid originates.

In embodiments, the synovial joint is located in a spine (cervical, thoracic, and lumbar), elbow, fingers, foot, wrist, hip, shoulder, or knee. For example, the synovial joint can comprise a facet joint. As used herein, the term facet joint can refer to joints which form from the superior and inferior articular processes of two adjacent vertebrae. For example, the facet joint can comprise a cervical facet joint, a thoracic joint, or a lumbar facet joint. As used herein, the term “cervical” can refer to the neck region of a spinal column or backbone. In embodiments, the cervical spine can comprise bones C1 to C7. As used herein, the term “thoracic” can refer to the upper and middle part of the spinal column comprising bones T1 to T12. As used herein, the term “lumbar” can refer to the vertebrae between the thoracic vertebrae and the sacrum. In embodiments, the lumbar spine can comprise bones L1 to L5.

In embodiments, the synovial fluid can leak from a synovial joint. For example, the synovial fluid can leak during physiologic gaping. As used herein, the term “physiologic gaping” can refer to a small opening of the facet joint during the endpoints of flexion or extension. For example, the capsule can be stretched and any defects from injury or degenerative weakening can allow for egress of the synovial fluid into the surrounding para-spinal musculature. As used herein, the term “para-spinal musculature” can refer to the muscles that support the back. As used herein, the terms “para-spinal muscles”, “erector spinae”, and multifidus muscles can be used interchangeably.

In embodiments, the synovial fluid leak can be caused by acute joint effusion. As used herein, the term “joint effusion” can refer to a swollen joint. For example, joint effusion can be caused by the movement of fluid into the soft tissues surrounding the joint.

In embodiments, the synovial fluid can leak from a synovial joint that is strained or injured. For example, the synovial capsule of the synovial joint can be injured. In embodiments, excessive axial load, rotation force, or repetitive tasks can lead to strain or injury to the fibro-cartilaginous joint capsule. This initial mechanical event can result in a separation or opening of the capsule thereby allowing egress of synovial fluid into the surrounding para-facet tissue such as the multifidus muscle and ligamentum flavum. Once the synovial fluid is extra-capsular it produces a secondary immune response.

In embodiments, the synovial fluid can leak from a synovial joint in a person who has a history of being sedentary. As used herein, the term “sedentary” can refer to a waking behavior characterized by energy expenditure less than or equal to 1.5 metabolic equivalents while in a sitting, reclining, or lying posture.

In some embodiments, the synovial fluid outside of the synovial joint originates from a tear or rupture from a synovial capsule.

In some embodiments, the synovial fluid outside of the synovial joint does not originate from a tear or rupture of a synovial capsule. For example, the synovial fluid outside of the synovial joint can originate from the rupture of a synovial cyst. For example, the synovial cyst generally arises in late-stage progression of leaky back syndrome.

In certain embodiments the synovial joint, synovial capsule of the synovial joint, and/or synovium of the synovial joint is inflamed (synovitis). As used herein, the term “inflamed” can refer to a part of the body being affected by inflammation. As used herein, the term “inflammation” can refer to a local response to cellular injury that is marked by capillary dilation, leukocytic infiltration, redness, heat, and pain and that can serve as a mechanism initiating the elimination of noxious agents and/or damaged tissue. The terms “inflammation” and “inflammatory response” can refer to the combined biological response of an individual's tissue to harmful stimuli such as injury, pathogens, viruses, damaged cells, or irritants. Inflammation and inflammatory response can include secretion of cytokines, such as inflammatory cytokines (i.e., cytokines produced primarily by active immune cells such as microglia and involved in the amplification of inflammatory responses). Exemplary inflammatory cytokines include, but are not limited to, IL-1, IL-6, TNF-a, IL-17, IL21, IL23 and TGF-β. Exemplary inflammation includes acute inflammation and chronic inflammation. As used herein, the term “acute inflammation” can be characterized by the classic signs of inflammation (swelling, hyperemia, pain, high fever, loss of function) resulting from tissue infiltration by plasma and leukocytes. Acute inflammation occurs as long as harmful stimuli are present and stops once the stimuli are removed and degraded or surrounded by scars (fibrosis). As used herein, the term “chronic inflammation” can refer to a condition characterized by ongoing concurrent active inflammation, tissue destruction, and attempts at repair. Chronically inflamed tissues are characterized by infiltration, tissue destruction, and recovery of mononuclear immune cells (monocytes, macrophages, lymphocytes and plasma cells), angiogenesis and fibrosis including symptoms.

Without wishing to be bound by theory, a subject's allergenicity and/or fibrogenesis can determine the inflammation.

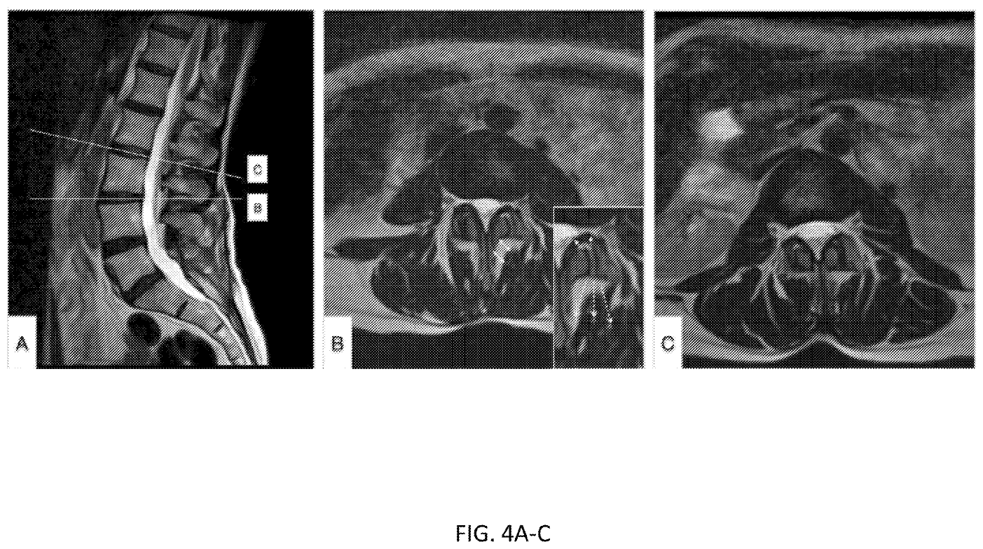

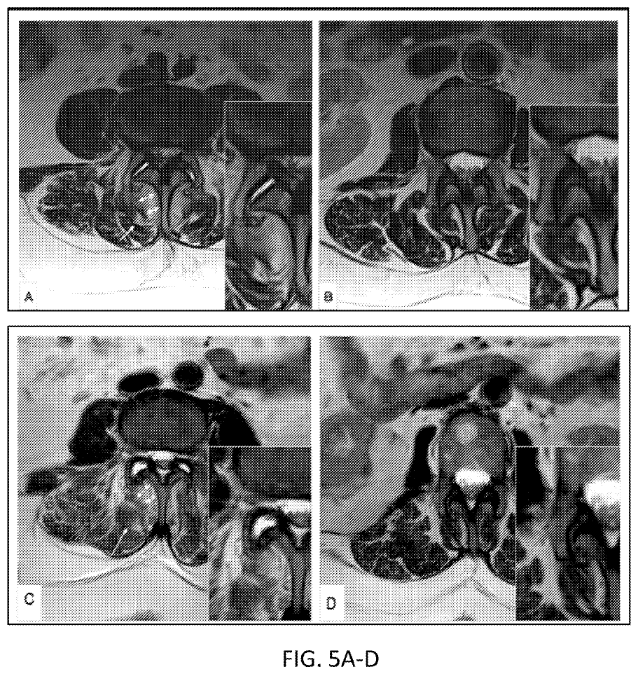

Without wishing to be bound by theory, leaky back syndrome can begin as an inflammatory process and if left untreated, can lead to degenerative disorders (FIG. 4A-C, FIG. 5A-D).

In some embodiments, the synovial fluid and/or components outside of the synovial joint originates from leakage from a synovial joint that is proximal to the detected synovial fluid. As used herein, the term “proximal” can refer to being situated close to a particular locale. In embodiments, the synovial fluid outside of the synovial joint inflames tissue surrounding the proximal synovial joint. For example, the tissue surrounding the proximal synovial joint comprises, dorsally: ligament flavum, dural surface, epidural fat, paraspinal musculature, and/or an exiting nerve root. For example, the tissue surrounding the proximal synovial joint comprises, ventrally: multifidus muscle and/or medial branch nerve.

In embodiments, the synovial fluid and/or components outside of the synovial joint are detected in paraspinal musculature or in underlying ligamentum flavum. As used herein, the terms “yellow ligament” and “ligamenta flavum” can be used interchangeably. In embodiments, ligamentum flavum can refer to paired ligaments which run between adjacent laminae of the vertebral bodies and are present from C2/3 to the sacrum.

In certain embodiments, detection of individual components in synovial fluid can serve as a proxy for detecting synovial fluid. For example, the individual components can comprise hyaluronic acid, lubricin, as well as Ig lambda chain C region, Serotransferrin, Immunoglobulin lambda variable 8-61, Immunoglobulin kappa variable 6-21, Apolipoprotein A-I Hemopexin, Alpha-2-HS-glycoprotein (Bennike et al., J. Proteome Res. 2014, 13, 4377-4387). In some embodiments, fragments of one or more components of synovial fluid (e.g., hyaluronic acid) can serve as a proxy for detecting synovial fluid.

In embodiments, the synovial fluid and/or components thereof outside of the synovial joint does not originate from extracapsular fluid. For example, the synovial fluid does not originate from transcellular fluid in the extracapsular fluid.

### Symptoms of Leaky Back Syndrome

In embodiments, symptoms of leaky back syndrome can comprise both acute and chronic back pain. As used herein, the term “back pain” can refer to discomfort occurring anywhere on the spine or back of a subject. For example, back pain can comprise acute back pain and chronic back pain.

As used herein, the term “acute back pain” can refer to back pain lasting less than about 1 hr, less than about 6 hrs, less than about 12 hrs, less than about 24 hrs, less than about 48 hrs, less than about 3 days, less than about 5 days, less than about 1 week, less than about 2 weeks, less than about 3 weeks, less than about 4 weeks, less than about 5 weeks, less than about 6 weeks, less than about 7 weeks, less than about 8 weeks, or less than about 3 months. In embodiments, symptoms of acute low back pain can comprise morning stiffness, paraspinal muscle spasms, posterior buttock pain, proximal leg pain.

As used herein, the term “chronic back pain” can refer to back pain lasting at least about 12 weeks, at least about 13 weeks, at least about 14 weeks, at least about 15 weeks, at least about 16 weeks, at least about 4 months, at least about 6 months, at least about 9 months, more than about 9 months, more than about 1 year, more than about 2 years, more than about 3 years, more than about 4 years, more than about 5 years, more than about 10 years, more than about 20 years, more than about 50 years, or more than about 100 years.

In embodiments, chronic back pain can comprise recurrent chronic back pain and progressive back pain. As used herein, the term “recurrent chronic back pain” can refer to repeated episodes of back pain with each episode lasting greater than 3 months. For example, symptoms of recurrent chronic back pain can comprise morning stiffness, paraspinal muscle spasms, posterior buttock pain, and proximal leg pain. In embodiments, recurrent chronic back pain can result in the development of activity dependent pain. As used herein, the term “activity dependent pain” can refer to pain which coincides with an activity. As used herein, the term “progressive chronic back pain” can refer to pain which originates from a constant cause. In embodiments, progressive chronic back pain can increase in severity over time. In embodiments, progressive chronic back pain symptoms can comprise activity dependent pain and morning stiffness.

In certain embodiments, a subject with leaky back syndrome may not have back pain.

### Non-Limiting Examples of Imaging Diagnostics

In embodiments, a subject with leaky back syndrome can have unremarkable radiographic presentation. In embodiments, the subject does not have remarkable radiographic presentation of lumbar degeneration. In embodiments, the subject does not have remarkable radiographic presentation of fatty infiltration of the multifidus muscle or significant degenerative changes of the facet joints. Instead, early stages of leaky back syndrome are characterized by an episode of moderate to severe axial back pain often described as morning stiffness and is relieved with exercise. Radiographic changes are minimal but can show some signs of increased thickness of the ligamentum flavum compared to normal controls and subtle loss of muscle striations in the multifidus muscle.

As used herein, the term “unremarkable” can refer to a diagnostic image that can refer to an image that does not demonstrate or prove the presence or existence of symptoms. As used herein, the ten “unremarkable” can refer to a diagnostic image that does not appear abnormal to a person of ordinary skill in the art. For example, a person of ordinary skill in the art can comprise a clinician. For example, the clinician is a physician, a physician associate, a nurse, a radiology technician, a resident physician, or a medical student. For example, the clinician is a radiologist.

In embodiments, the subject has unremarkable lumbar radiography. For example, lumbar degeneration is not remarkable based upon standard radiographical techniques.

In embodiments, the subject can have radiographic indication of facet joint capsule thickening or hypertrophy of the entire joint. In embodiments, a subject can have bouts of synovial fluid released from synovial joints. In some examples, the synovial joints can heal (e.g., scarring, scabbing, plugs can be present), but later release synovial fluid.

In embodiments, the subject does not have fatty infiltration of the multifidus muscle. For example, radiographical techniques do not show remarkable fatty infiltration of the multifidus muscle. In some embodiments, the stage of the disease is so early in the subject, that fatty infiltration of the multifidus muscle has not yet occurred. Without wishing to be bound by theory, fatty infiltration will occur in later stages of disease progression.

In embodiments, the synovial fluid and/or components of synovia fluid outside of the synovial joint can be detected by imaging. For example, the imaging can comprise computed tomography, positron emission tomography, magnetic resonance imaging (MRI), SPECT, photo acoustic imaging. two photon or fluorescent imaging or ultrasonography.

Aspects of the invention are drawn towards radiopharmaceutical tags for the identification and diagnosis of leaky back syndrome. Without wishing to be bound by theory, a radiopharmaceutical tag can be used to identify localized extra-capsular synovial fluid leaks. In embodiments, the radiopharmaceuticals are diagnostic or therapeutic radioactive components comprising a radionuclide, a linker/chelator, and a ligand or vector molecule for target localization (Vermeulen et al., Semin. Nucl. Med. 49:339-356, 2019). The vector molecular is a part of the radiopharmaceutical as it is response for the selective interaction with the target tissue or fluid. Common vectors used are small molecules ranging from amino acids, fatty acids. Peptide and proteins including monoclonal antibodies can also be considered as they provide an ability to target specific epitopes and components of the inflammatory pathway as well as bind to receptors. The radionucleotide are elements with and excess of nuclear energy with a broad variety of half-lives and energies of the radionucleotide to target and visualize specific target tissue or fluid. Common radionucleotides comprise ^{99m}Tc and ¹⁸F among others that are available.

Aspects of the invention are drawn towards biomarkers for the detection of Leaky Back Syndrome. In embodiments, the biomarkers can comprise components of synovial fluid. For example, the components can comprise hyaluronic acid or a fragment thereof. As used herein, the terms “hyaluronic acid” and “hyaluronan” can be used interchangeably. In embodiments the hyaluronic acid can comprise low molecular weight (LMW) hyaluronic acid. As used herein, the term “low molecular weight” can refer to a molecule with a molecular weigh of about 500 Da or less. In embodiments, the hyaluronic acid fragment can comprise a hyaluronic acid oligomer, a hyaluronic acid monomer, D-glucuronic acid, N-acetyl-D-glucosamine, or a combination thereof. In some embodiments, the hyaluronic acid and/or fragments thereof may not originate from synovial fluid.

Aspects of the invention are drawn towards antibodies used in the detection of Leaky Back Syndrome. In embodiments, an antibody can be used to detect synovial fluid outside of a synovial joint. For example, the antibody can detect a component of the synovial fluid. In embodiments, components of the synovial fluid detectable by the antibody can comprise hyaluronic acid, proteoglycan 4, surface-active phosphor lipids, CXCL1, CXCL5, IL-6, IL-8, IL-10, TNFα Ig lambda chain C region, Serotransferrin, Immunoglobulin lambda variable 8-61, Immunoglobulin kappa variable 6-21, Apolipoprotein A-I Hemopexin, and/or Alpha-2-HS-glycoprotein. (Bennike2014).

In embodiments, the antibody can comprise a monoclonal antibody, a bispecific antibody, an scFv, or a fragment thereof. In embodiments, the antibody is attached to a detectable moiety. For example, the detectable moiety can comprise a radionucleotide, fluorescent molecule, bioluminescent molecule, and/or light-emitting molecules. In embodiments, these moieties can be detected by imaging methods. For example, a biomarker can be localized with a radiotracer linked to a mAb.

### Methods Described Herein

Aspects of the invention are drawn towards methods of diagnosing early stages of degenerative join disease comprising detecting synovial fluid and/or components thereof outside of a synovial joint in an animal (FIG. 15). The animal can be a human.

Aspects of the invention are drawn towards methods of identifying a subject at risk for developing inflammation of tissue surrounding a facet joint comprising detecting synovial fluid and/or components outside of the facet joint. In embodiments, the synovial fluid is in proximity to the fact joint. In embodiments, the subject does not have degenerative joint changes. For example, the subject does not have degenerative lumbar changes. In embodiments, the subject does not have fatty infiltration in the multifidus muscle. In some embodiments, the subject does not have a synovial cyst associated with the facet joint. In some embodiments, the subject does not have intervertebral disc disease. In some embodiments, the subject presents with pain. For example, the pain can be lumbar pain. In some embodiments, the pain is acute pain. In some embodiments, the acute pain is recurrent. In embodiments, the synovial fluid and/or components can be detected using a substance that binds to a component of the synovial fluid. For example, the substance comprises an antibody. In embodiments, the substance comprises an antibody. For example, the antibody is attached to a detectable moiety. For example, the detectable moiety can be detected by imaging methods.

Aspects of the invention are drawn towards a method for detecting synovial fluid and/or components thereof surrounding a synovial joint in a human patient with pain comprising administering to the human patient a substance that can bind a component of extracapsular synovial fluid; detecting the substance bound to the component of extracapsular synovial fluid in the human patient. In embodiments, the pain comprises acute lumbar pain. In embodiments, the antibody is attached to a detectable moiety.

Aspects of the invention are drawn towards a method of identifying a subject at risk for developing degenerative lumbar disease comprising detecting extracapsular synovial fluid and/or components thereof in proximity to a synovial joint. In embodiments the extracapsular synovial fluid and/or components is detected in ligament flavum, dural surface, epidural fat, paraspinal musculature, an exiting nerve root, multifidus muscle, and/or a medial branch nerve.

Aspects of the invention are drawn towards a method for identifying a human at risk for developing degenerative lumbar disease, comprising detecting inflamed tissue surrounding a synovial joint. In embodiments, the tissue comprises ligament flavum, dural surface, epidural fat, paraspinal musculature, an exciting nerve root, multifidus muscle, and/or a medial branch nerve. In embodiments, the inflamed tissue is detected using a blood test. In embodiments the blood test detects inflammatory markers. For example, inflammatory markers can comprise hyaluronic acid, proteoglycan 4, surface-active phosphor lipids, CXCL1, CXCL5, IL-6, IL-8, IL-10, TNFα Ig lambda chain C region, Serotransferrin, Immunoglobulin lambda variable 8-61, Immunoglobulin kappa variable 6-21, Apolipoprotein A-I Hemopexin, and/or Alpha-2-HS-glycoprotein.

Aspects of the invention are drawn towards a method for identifying a subject at risk for developing degenerative lumbar disease comprising detecting scarring, scabbing, and/or plugs in a synovial joint. In embodiments, the method comprises radiology.

Aspects of the invention are drawn towards a method for identifying a subject at risk for developing degenerative lumbar disease, comprising detecting inflammatory molecules, or elevated levels thereof, in synovial fluid of a lumbar synovial joint. In embodiments, the synovial fluid used in the method is obtained by arthrocentesis.

Aspects of the invention are drawn toward a method for identifying a subject at risk for developing degenerative lumbar disease comprising detecting inflammatory molecules, or elevated levels thereof, in a tissue sample obtain from tissue in proximity to a lumbar synovial joint.

In some embodiments of the methods disclosed herein, hyaluronic acid (HA) and/or fragments of HA can be detected. In some embodiments, an HA fragment can be an HA that is acted on by a hyaluronidase. In some embodiments, an HA fragment can be a high molecular weight HA (HMW) that has been cleaved by a hyaluronidase. In some embodiments, an HA fragment can be a low molecular weight HA (LMW). In some embodiments, an HA fragment can be a low molecular weight (LMW) hyaluronic acid, an oligomer of hyaluronic acid, a monomer of hyaluronic acid, D-glucuronic acid, N-acetyl-D-glucosamine, or a combination thereof. HMW and LMW are described elsewhere herein.

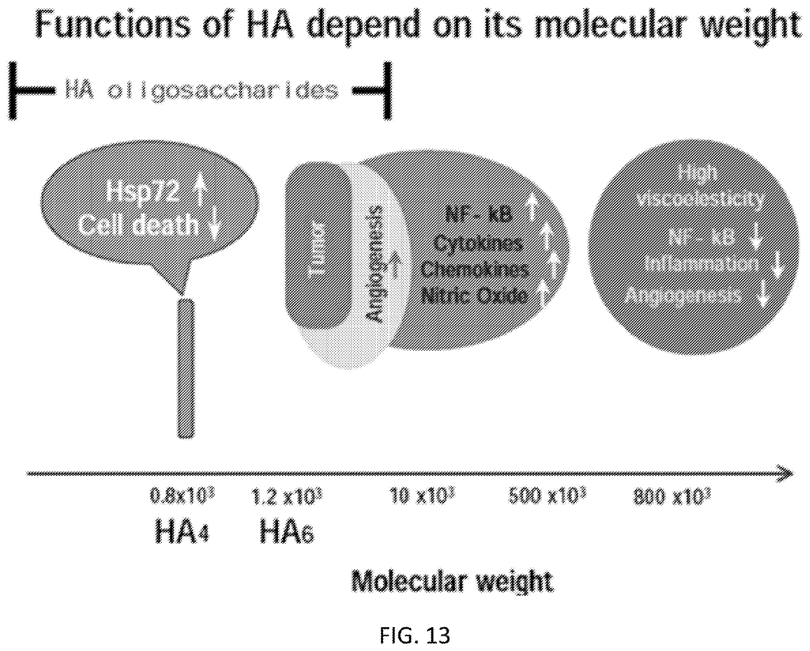

In some embodiments of the methods disclosed herein, a change in the distribution of HA and/or HA fragment sizes, as compared to a control, can be detected and used as an indicator of leaky back syndrome. For example, a distribution of HA fragment sizes found in tissue that is inflamed (e.g., the HA originated from synovial fluid leaked from a facet joint, leaked into surrounding tissue, and has been partially degraded by hyaluronidases) can have a lower mean size than a distribution of HA fragment sizes found in synovial fluid that has not leaked from a synovial joint. In some embodiments, a size distribution of HA fragments from leaked synovial fluid may be otherwise different from a size distribution of HA fragments that has not leaked from a synovial joint (e.g., different median, mode, standard deviation, and the like). Some sizes of HA fragments can contribute to inflammation (FIG. 13).

### Non-Limiting Examples of Leaky Back Syndrome Treatments

Aspects of the invention are drawn towards treatments for leaky back syndrome. In embodiments, treatments for leaky back syndrome can comprise immunologic therapies, pharmaceutical therapies, nanotherapeutics, and stem cell therapy. In embodiments, pharmaceutical therapies can comprise anti-inflammatories, and antifibrotics. In embodiments, immunologic therapies can comprise monoclonal antibodies, immune system modulators, immune checkpoint inhibitors, and adoptive cell therapies. For example, the monoclonal antibody can comprise adalimumab. In embodiments, nanotherapies can comprise small molecules and peptides that target and block specific components of the inflammatory pathway. In embodiments, stem cell therapies can comprise mesenchymal stromal cell-derived, extracellular vesicles and/or exosomes. In some embodiments, treatments can be directed toward stopping synovial fluid leakage from the synovial joint.

In embodiments, the therapeutic can reduce a subject's inflammatory response to leaked synovial fluid. In embodiments, the therapeutic can enhance the body's response to synovial fluid. Without wishing to be bound by theory, enhancing the body's response to synovial fluid can increase “scab” fibrogenesis. In embodiments, a measurement of success of therapeutics described herein can be an improved quality of life.

In embodiments, the administration of TNFα and IL-6 inhibitors either systemically or via local injection to the site of leakage can be used as therapeutics for leaky back syndrome. Similar to how these agents have been used in spondyloarthropathies such as ankylosis spondylitis, without wishing to be bound by theory, the extra-capsular synovial fluid leakage activates downstream activation of TNFα and thus the development of biologics that specifically target this inflammatory pathway can be used as therapeutics (Furst and Louie, Arthritis Research & Therapy (2019) 21:135).

In some embodiments, a therapy can be a sealant. For example, the sealant can plug areas of synovial fluid leakage across fibrous joint capsules.

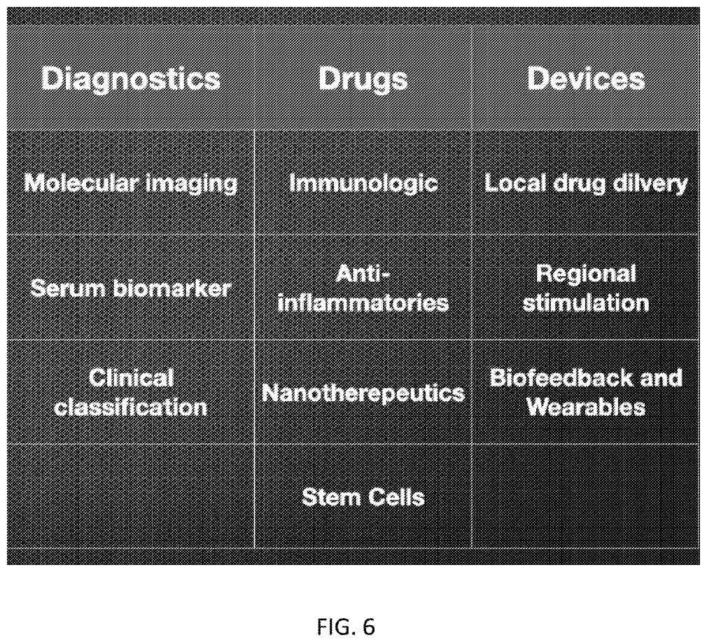

FIG. 6 illustrates examples of various treatments.

### Non-Limiting Examples of Devices of the Disclosure

Aspects of the invention are drawn towards devices for treating, preventing, and diagnosing leaky back syndrome. In embodiments, devices can comprise local drug delivery devices, regional stimulation devices, biofeedback devices, and wearable devices. For example, the devices can comprise devices that simulate the spinal cord. For example, the device is a neuromodulator. For example, the wearable sensor can detect the downstream product of synovial fluid and alert the user of the changes.

### Kits

The invention also provides for a kit for using any of the various diagnostics described herein. For example, a kit of the disclosure can comprise instructions for diagnosing leaky back syndrome, therapeutics for treating leaky back syndrome, and the like.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1—Leaky Back Syndrome: Description of the Pathophysiologic, Radiographic and Clinical Manifestations of Facet Joint Synovial Fluid Leakage and Treatment and Diagnostic Innovations

Introduction: Chronic low back pain is a widespread problem affecting millions of adults in the United States resulting in significant impairment of quality of life and lost productivity (1,2). While inflammation is accepted as contributing to the symptoms of both acute and chronic low back pain, identification of an upstream biomarker that activates this the secondary inflammatory response has been elusive thus limiting the development of effective diagnostics and therapeutics. Herein we describe a pathophysiologic process of synovial fluid leakage from the lumbar facet joints resulting in clinical and radiographic changes known as leaky back syndrome. The process of synovial fluid leakage is responsible for activating the downstream inflammatory response resulting in a constellation of clinical and radiographic manifestations of acute and chronic axial back pain, respectively. Here we present in-situ evidence of the phenomena demonstrating synovial fluid leakage, describe the clinical and radiographic correlations believed to be associated with leaky back syndrome and diagnostic and therapeutic innovations based on the existence of this model of disease.

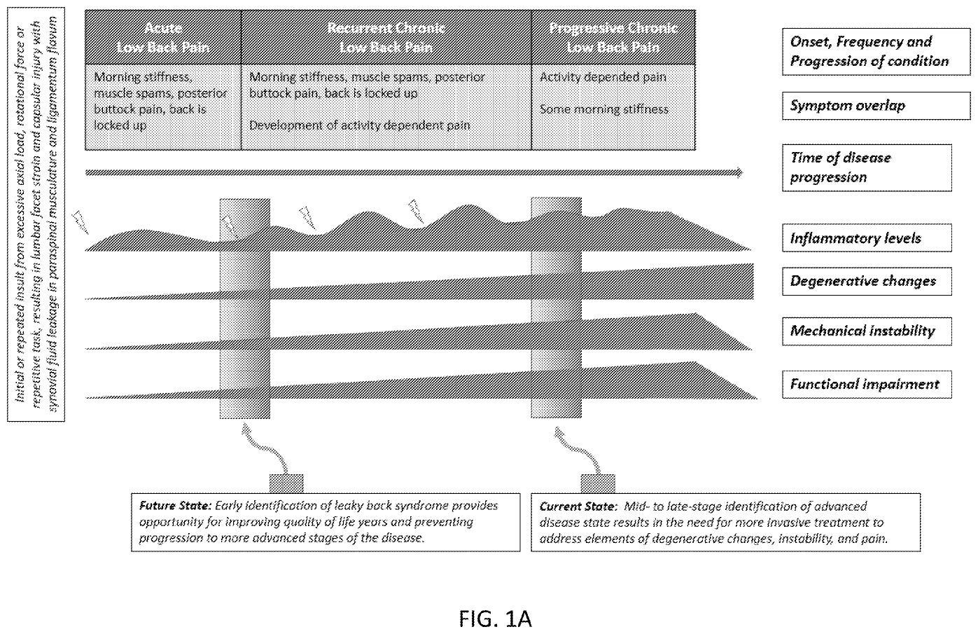

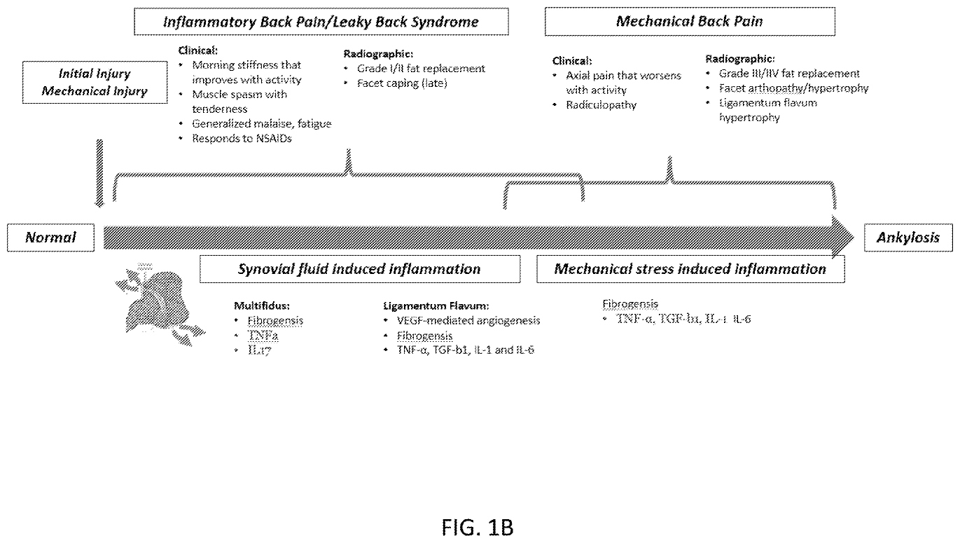

Background: Clinicians that treat patients with back pain attribute many of the symptoms associated with the episode to be related to a secondary inflammatory response. There is no available biomarker that can be used to monitor the symptoms of back pain in the acute state and explain the downstream degenerative changes seen in the chronic state. Thus, clinicians attempt to correlate symptoms with the clinical exam and radiographic changes to determine the optimal treatment options. Unfortunately, especially in acute episodes of isolated back pain, it has been reported that perhaps as many as 85% of patients with isolated low back pain cannot be diagnosed with a precise pathoanatomic abnormality (NEJM, 2001). In the event there are changes seen on imaging, the specificity of a radiographic change as a marker of symptomatic spine disease has been put into question as several studies have demonstrated that many asymptomatic individuals can harbor similar radiographic changes. Therefore, the identification of a biomarker that actives the inflammatory response and mediates the symptoms of low back pain especially in the early stages can provide the opportunity for improving quality of life years and preventing progression to more advanced stages of the disease (FIGS. 1A and 1B).

Methods: We use clinical and radiographic observations made after treating patients with acute and chronic back conditions along with video footage demonstrating in-situ the mechanism of synovial fluid leakage to describe a condition called leaky back syndrome. We can corroborate this model using data from peer-reviewed literature that supports our model of extra-capsular synovial fluid leakage as the genesis of downstream inflammation in conditions affecting the spine and major joint structures. This model serves as the platform for the development of treatments including but not limited to radiopharmaceuticals for diagnostics and disease state monitoring, nano-therapeutics and immunological treatment for symptom relief and disease modification, implantable devices and injectables for disease management and symptom relief as well as public health strategies to reduce the prevalence of conditions associated with chronic low back pain.

### Mechanism of Leaky Back Syndrome:

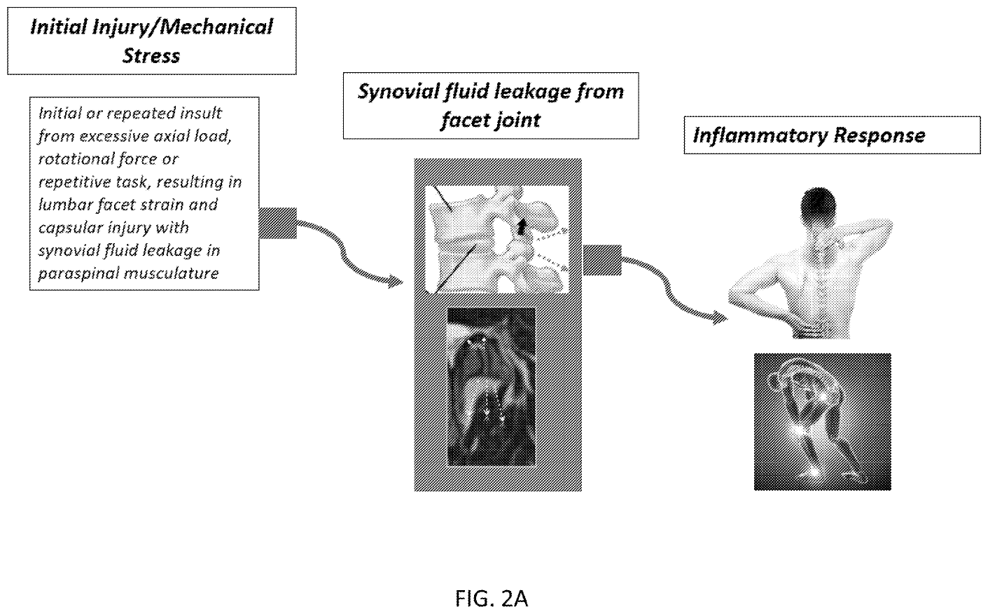

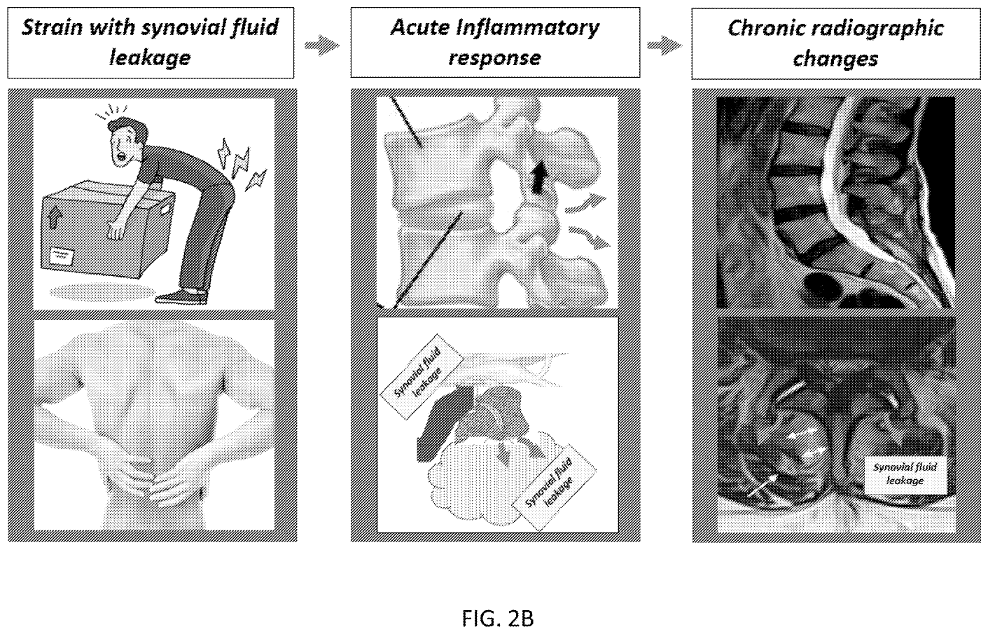

We provide compelling evidence of a mechanism which demonstrates the leakage of synovial fluid from the zygapophyseal joints of the spine into the surrounding paraspinal musculature (FIGS. 2A and 2B). The leakage of synovial fluid into the surrounding paraspinal musculature and, without wishing to be bound by theory, the underlying ligaments flavum results in mediating common symptoms associated with acute and chronic back pain episodes such as morning stiffness, paraspinal muscle spasm and proximal leg pain.

Without wishing to be bound by theory, the leakage of synovial fluid is greatest during physiologic gaping which is the small opening of the facet joint during the endpoints of flexion or extension (Tisher et al 2006). At this endpoint the capsule is stretched and any defects either from injury or ongoing degenerative weakening will allow egress of the synovial fluid into the surrounding parka-spinal musculature.

The lumbar facet joints are zygapophyseal joints that are part with each lumbar vertebra. The joint pair provide stability to the spine and help off-load axial loads. The joint is composed of two have of half moon-shaped bones with an intervening synovial membrane. This membrane produces synovial fluid with visco-elastic properties enhancing axial load sharing during weight bearing activities. While there is literature supporting the content of osteoarthritic changes affecting the composition of the synovial fluid there has not been any reports directly linking synovial fluid leakage both into the paraspinal muscular as well as the ligaments flavum as the primary process mediating both the acute and chronic inflammatory pathways which then trigger many of the initial symptoms of axial back pain and in the chronic state results in more advanced radiographic changes of degeneration of the intra and extra-spinal posterior elements and the para-facet tissues.

Without wishing to be bound by theory, the leakage of synovial fluid from the zygapophyseal joints as the central inflammatory mediator that produces many of the symptoms associated with episodes of isolated acute low back pain. Without wishing to be bound by theory, the leakage of synovial fluid into the surrounding paraspinal musculature and intraspinal canal results in the constellation of symptoms associated with forms of acute and chronic back pain episodes. The presence of synovial fluid outside of its native can act as the mediator of the inflammatory response that is responsible for the clinical symptoms associated with acute axial back pain episode and the subsequent radiographic changes of degeneration in the chronic state.

The synovial fluid composition consists of a number components, including an ultrafiltrate. In several disease states there is up regulation of inflammatory molecules. The release of biologically active soluble mediators into the surrounding paraspinal musculature leads to the local activation of inflammatory pathways both acute and chronic mediating many of the symptoms of associate with back pain episodes.

### Inflammatory Response Following Extra-Capsular Synovial Fluid Leakage and Response of the Parafacet Structures

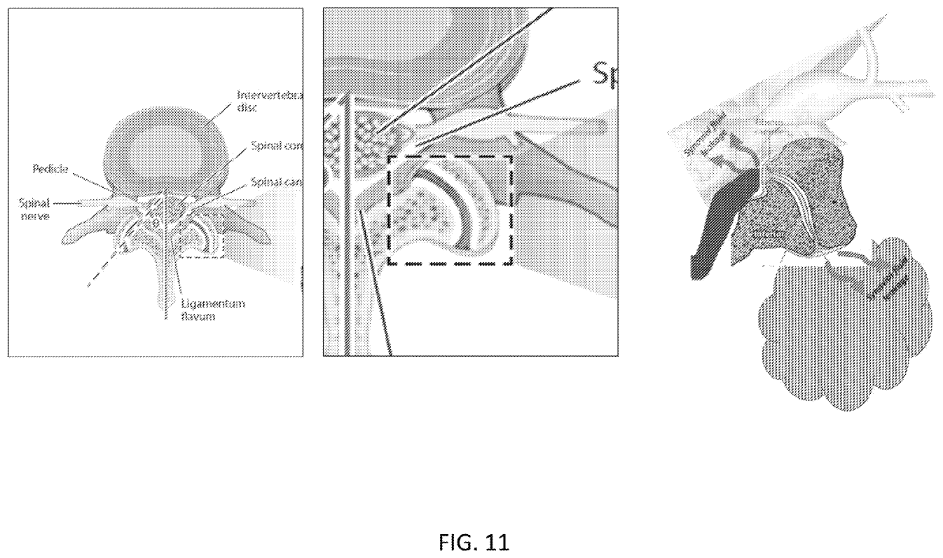

In some patients the presence of extra-capsular synovial fluid activates a robust inflammatory response within the structures surrounding the facet joint. These tissues include dorsally the ligament flavum, dural surface, epidural fat and paraspinal musculature as well as exiting nerve roots. Ventral to the facet joint the tissue included multifidus muscle and the medial branch nerve. (FIG. 11)

Immediately following the leakage of synovial fluid, and without wishing to be bound by theory, a sequence of local tissue inflammatory responses that depending on the patient's ability to modulate anti- and pro-inflammatory pathways.

TABLE 1
Table 1 shows non-limiting, exemplary inflammatory responses following the leakage of synovial fluid.

| | | | | | | |
|---|---|---|---|---|---|---|
| Para-facet | Paraspinal | Ligamentum | Exiting roots | Thecal sac, | Epidural fat | Medial branch |
| Tissue | Musculature | Flavum | | (dural sac) | | nerves |
| Inflammatory | Fibrogenesis | Fibrogenesis | Angiogenesis | Migraine- | — | Nociceptor |
| pathway | | | | like | | activation |
| Radiographic | Fat | Hypertrophy | — | Thinning | hypertrophy | — |
| Finding d/t | replacement | | | | | |
| chronic LBS | | | | | | |
| Without being | Anti TNF | Stem Cell | Anti-VEGF | Betablockers | Mesenchymal | — |
| bound by theory, | alpha | | | | stem cells | |
| treatment target | | | | | | |

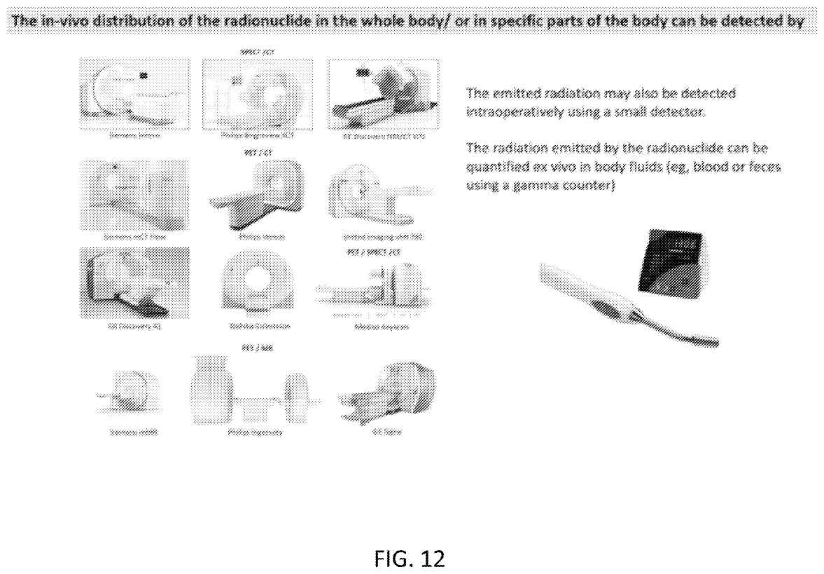

Certain molecular imaging methods to detect extra capsular synovial fluid leakage. One such method involves the in vivo distribution of radionuclide identifying extra-capsular synovial fluid detected by the following modalities. (FIG. 12)

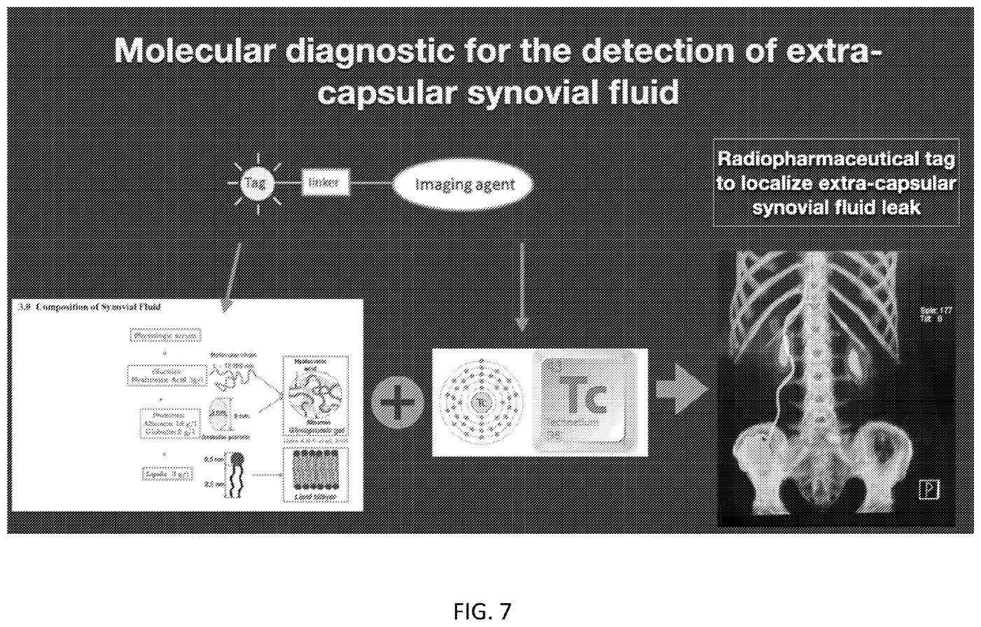

An example radiopharmaceutical targeted to detect extra capsular synovial fluid leakage is shown (FIG. 7).

TABLE 2
Table 2 provides, without wishing to be bound by theory,
example molecular imaging targets to diagnose LBS.

| Inflammatory | | |
|---|---|---|
| Molecule | Synovial Fluid | Articular Cartilage |
| IL-6 | Proteoglycan 4 | Small leucine-rich |
| | | proteoglycans, decorin, |
| | | biglycan, aspirin |
| VEGF | Hyaluronic acid | |
| TNF-a | Surface active | |
| | phosphor lipids | |

Serum biomarkers to detect presence of LBS—post inflammatory siRNA, DNA.

### REFERENCES

1. Frymoyer J W. Back pain and sciatica. N Engl J Med 1988; 318:291-300.
2. Lurie J D, Gerber P D, Sox H C. Clinical problem-solving. A pain in the back. N Engl J Med 2000; 343:723-6.
3. Ralf Weiskirchen, Sabine Weiskirchen, Frank Tacke, Organ and tissue fibrosis: Molecular signals, cellular mechanisms and translational implications, Molecular Aspects of Medicine, Volume 65, 2019, Pages 2-15.
4. Prete P. E, Gurakar-Osbome A, Kashyap M. L, Synovial fluid lipoproteins: review of current concepts and new directions, Seminar Arthritis Rheum, 1993.
5. Simkin P. A, Arthritis and Allied conditions, A text book of Rheumatology, Editors D. J. McCarty& W. J Koopman, Lea & Febiger, Philadelphia, 1993.
6. Weinberger A, Simkin P. A, Plasma proteins in synovial fluids of normal human joints, Semin Arthritis Rheum, 1993.
7. Alice Mae Pendleton, Bio fluid lubrication for artificial joints, Dissertation, 2008.
8. Swann D A, Silver F H, Slayter H S, Stafford W, and Shore E, The molecular structure and lubricating activity of lubricin isolated from bovine and human synovial fluids, Biochem J 1985.
9. Schmid T, Lindley K, Su J, Soloveychik V, Block J, Kuettner K, and Schumacher B, Superficial zone protein (SZP) is an abundant glycoprotein in human synovialfluid and serum, Trans Orthop Res Soc 2001.
10. Ogston A G and Stanier J, The physiological function of hyaluronic acid in synovial fluid: viscous, elastic and lubricant properties. J Physiol 1953.
11. Mazzucco D, Scott R, and Spector M, Composition of joint fluid in patients undergoing total knee replacement and revision arthroplasty: correlation with flow properties, Biomaterials 2004.
12. Sutovsky, Juraj, et al. “Cytokine and chemokine profile changes in patients with lower segment lumbar degenerative spondylolisthesis.” International Journal of Surgery 43 (2017): 163-170.
13. Igarashi, Akira, et al. “Inflammatory cytokines released from the facet joint tissue in degenerative lumbar spinal disorders.” Spine 29.19 (2004): 2091-2095.
14. Anderson, Brad, et al. “Paraspinal Muscle Health is Related to Fibrogenic, Adipogenic, and Myogenic Gene Expression in Patients with Lumbar Spine Pathology.” BMC Musculoskeletal Disorders 23.1 (2022): 1-11.

### Example 2

### Example 2: Back Pain Biomarker

### Problem

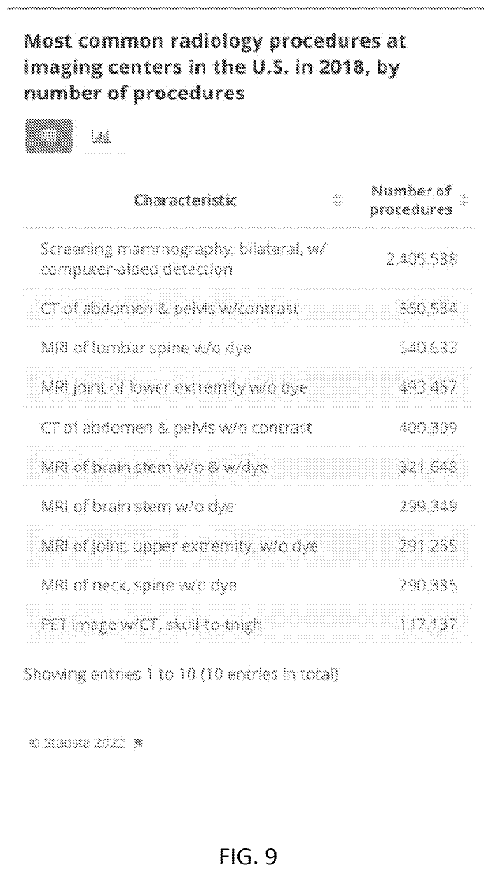

Low back pain is a common and often debilitating condition
The underlying source of back pain is often not known
Discovery of the biomarker that leads to low back pain will revolutionize diagnosis and treatment
Most common radiology procedures at imaging centers in the U.S. in 2018, by number of procedures (FIG. 9).
Palmer et al., Clin. Ep. Immunol. (1985) 59, 529-538 shows examples of synovial membrane identified with monoclonal antibodies.
Paraspinal Fibrogenesis in LBS

There is increasing evidence indicating inflammation as one of the most central events in fibrosis. See, e.g., Ralf Weiskirchen, Sabine Weiskirchen, Frank Tacke. Organ and tissue fibrosis: Molecular signals, cellular mechanisms and translational implications. Molecular Aspects of Medicine, Volume 65, 2019, Pages 2-15.
Fibrosis denotes excessive scarring, which exceeds the normal wound healing response to injury in many tissues. Although the extracellular matrix deposition appears unstructured, disrupting the normal tissue architecture and subsequently impairing proper organ function, fibrogenesis is an orchestrated process determined by defined sequences of molecular signals and cellular response mechanisms. Persistent injury and parenchymal cell death provokes tissue inflammation, macrophage activation and immune cell infiltration.
   Stem cell Treatment
Increasing evidence has shown that inflammation is one of the most central events in fibrosis [7,8]. As a profibrotic cytokine, transforming growth factor β1 (TGF-β1) has been found to be associated with fibrosis in many organs and tissues [9,10]. Previous studies have found upregulated TGF-β1 expression levels in the hypertrophied LF of LSS patients [11, 12]. The increased synthesis of extracellular matrix (ECM) proteins after stimulation by TGF-β1 expression in LF cells [13] was observed, elucidating that the formation and accumulation of inflammatory.
Igarashi et al., Inflammatory cytokines released from the facet joint tissue in degrative lumbar spinal disorders, SPINE, Volume 29, Number 19, pp. 2091-2095, © 2004, Lippincott Williams & Wilkins, Inc.
Sutovsky et al., Cytokine and chemokine profile changes in patients with lower segment lumbar degenerative spondylolisthesis, International Journal of Surgery, 43, (2017), 163-170.

### Stem Cell Therapy to Mediate Inflammatory Response to Leaky Back Syndrome.

Despite the numerous studies explored the mechanisms of LF fibrosis at the molecular and cellular levels, the exact mechanism remains unknown. Without wishing to be bound by theory, pathophysiologic stimuli such as mechanical stress, aging, obesity, and some diseases are the causative factors.
Sun et al., Ligamentum flavum fibrosis and hypertrophy: Molecular pathways, cellular mechanisms, and future directions, FASEB J. 2020 August; 34(8):9854-9868.
Ma et al., Amelioration of ligamentum flavum hypertrophy using umbilical cord mesenchymal stromal cell-derived extracellular vesicles, Bioactive Materials, 19, (2023), 139-154.

### Example 3

### Example 3: Hyaluronan and Synovial Fluid

We have identified that extracapsular synovial fluid leakage can result from a facet capsule sub-failure and can be associated with symptoms of inflammatory back pain. The inflammatory response from the presence of extracapsular synovial fluid and its components can result in varying degrees of muscle stiffness. The presence of these symptoms can be detected and quantified before advanced degenerative changes are evident radiographically. This logic indicates that the the presence of extracapsular synovial fluid is the local, biological feedback that activates robust inflammatory pathways that have been evolutionary conserved to promote facet capsule healing when the human spine is confronted with a capsular sub-failure resulting in mechanical instability. In other words, the presence of extracapsular synovial fluid within the para-spinal tissue is the biofeedback fluid that ignites the molecular machinery responsible for reinforcing the spinal elements to restore stability and neutral zone function and is often associated with varying degrees of inflammatory back pain symptoms.

Without wishing to be bound by theory, components of synovial fluid, including hyaluronic acid, also called hyaluronan (HA), upon leakage from synovial joints, can be degraded. In some embodiments, the HA can interact with the extra-cellular matrix of the para-facet tissue and multifidus muscle resulting in HA degradation. These degradation products in can be responsible for activating an inflammatory response within the para-facet tissues including the facet capsule, multifidus muscle, ligamentum flavum and peripheral nervous systems. This multifaceted signaling across various spinal subsystems tissues can result in an interplay of clinical, radiographic, and inflammatory patterns and can be used as markers of the early spinal instability.

### Structure and Distribution of Hyaluronan:

Hyaluronan is a glycosaminoglycan (GAG) that is unbranched, non-sulfated, and not covalently attached to a protein core. It is a large molecule composed of repeating disaccharide units of D-glucuronic acid and N-acetyl-D-glucosamine (Hascall and Laurent, 1997). The sizes of HA polymers can range from 5000 to 20,000,000 Da in vivo, and the functions of HA can be dictated by their size (Zhu, 2019). In its native form, HA is a large molecule and can be referred to as a high-molecular weight HA (HMW-HA). The HA in synovial fluid is typically of a very high molecular weight, often in the millions of Daltons. The high molecular weight of synovial fluid HA can contribute to its viscoelastic properties. This is related to HMW-HA exhibiting a random coil structure that can expand in aqueous solutions and thus contributes to its viscoelastic properties in synovial joints and the vasculature (Scott and Heatley, 2002; Girish and Kemparaju, 2007; Toole 2004).

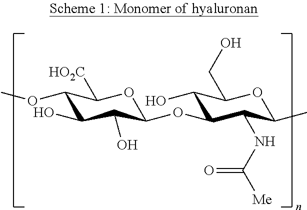

TABLE 3
Distribution, Molecular Weight, Concentration, and Unique
Properties of Hyaluronic Acid in Various Tissues

| | Molecular | | | |
|---|---|---|---|---|
| | Weight | | Turnover | Unique |
| Tissue | (Daltons) | Concentration | Rate | Properties |
| Synovial | 6-7 million | 2-3 mg HA/ml | Rapid | High |
| Fluid: | | | (several | molecular |
| Knee | | | times per | weight and |
| | | | day) | concentration |
| | | | | contribute to |
| | | | | viscoelasticity, |
| | | | | lubrication, |
| | | | | and shock |
| | | | | absorption. |
| | | | | Interacts with |
| | | | | lubricin. |
| Skin | 100,000-1 | 400-500 μg | Moderate | Contributes to |
| | million | HA/gtissue | | skin hydration |
| | | | | and elasticity. |
| | | | | Involved in |
| | | | | wound healing. |
| Vitreous | 2 million | 200 μg/ml | Slow | Provides |
| Humor | | | | structural |
| (Eye) | | | | support to the |
| | | | | eye |

Hyaluronan is ubiquitously distributed throughout the body, playing diverse and essential roles in various tissues (Table 3). It is a component of the extracellular matrix of many tissues, contributing to structural support, tissue hydration, and cell function. In synovial fluid, it is present at high concentrations (Table 3), where it plays a role in providing viscoelasticity, lubrication, and shock absorption (Fraser et al., 1997). In skin, it is involved in maintaining hydration and elasticity and plays an important part in wound healing. HA is also found in the vitreous humor of the eye, where it provides structural support. The concentration, molecular weight, and turnover rate of HA vary across these tissues, reflecting its versatile functions in the human body.

### Intra-Capsular Synovial Fluid and Hyaluronan:

The synovial fluid is a highly viscous liquid that fills the cavities of joints and serves several key functions. As a major component of synovial fluid, HA plays a role in the functions listed below.

Lubrication: HA is responsible for the lubricating properties of synovial fluid, which is important for protecting joints from wear and tear during movement (Fraser et al., 1997).

Shock Absorption: The high-molecular-weight HA in the synovial fluid provides shock absorption, safeguarding the joint during high impact activities (Fraser et al., 1997).

Nutrient Transport: HA plays a role in transporting nutrients between the synovial fluid and the articular cartilage, a crucial function given that the cartilage is avascular and relies on the synovial fluid for its nutrient supply (Fraser et al., 1997).

Hyaluronan Synthesis: Within the synovial joint, HA is synthesized by membrane-bound hyaluronan synthases (HAS1, HAS2, and HAS3). These glycosyltransferases catalyze the addition of uridine diphosphate (UDP)-linked N-acetylglucosamine and UDP-linked glucuronic acid to the growing HA chain at the non-reducing end, thus releasing the newly synthesized HA into the extracellular space (Prehm, 1984; DeAngelis, 1999; Itano and Kimata, 2002). Hyaluronic acid (HA) within synovial fluid has unique properties that distinguish it from HA in other tissues. In some embodiments, this can be used to distinguish HA and/or fragments thereof from synovial fluid from HA that originates elsewhere.

High Molecular Weight: The HA in synovial fluid is typically of a very high molecular weight, often in the millions of Daltons. This can be much larger than the HA found in most other tissues. The high molecular weight of synovial fluid HA contributes to its unique viscoelastic properties.

Viscoelasticity: HA gives synovial fluid its characteristic viscoelasticity, which allows it to act as both a lubricant and a shock absorber in the joint. When the joint is at rest, synovial fluid is thick and gel-like, helping to maintain joint stability. When the joint is moving, the synovial fluid becomes more liquid, allowing for smooth, frictionless movement.

Concentration: The concentration of HA in synovial fluid can be higher than in other tissues. This high concentration contributes to the fluid's ability to fill the joint space and provide a cushioning effect.

Turnover Rate: The turnover rate of HA in synovial fluid can be rapid, with the entire HA content of the joint being replaced several times a day. This is faster than the turnover of HA in most other tissues.

Interaction with Other Molecules: In synovial fluid, HA interacts with other molecules such as lubricin, a protein that also contributes to the lubricating properties of the fluid. This interaction is unique to the joint environment.

Inflammation and Healing: In conditions such as osteoarthritis and rheumatoid arthritis, metabolism of HA in the synovial joint can be altered. The molecular weight of HA can decrease, leading to a reduction in the viscosity of the synovial fluid, and can cause increased joint pain and/or decreased function. Fragments of HA can stimulate inflammation, recruiting immune cells to the joint and exacerbating the disease process (Volpi et al., 2009). The balance between synthesis and degradation of HA is important for maintaining the homeostasis of synovial fluid within the joint. Alterations in these processes, either in the activity of HA synthases or hyaluronidases, can contribute to joint diseases like arthritis (Volpi et al., 2009).

HA Degradation: Hyaluronan degradation is a complex process involving various enzymes and mechanisms with a proposed model of HA degradation involving the following steps:

Initiation: The degradation of HA can be initiated by different stimuli, such as tissue injury, inflammation, or enzymatic activation. These stimuli can trigger the release of specific enzymes or activate existing enzymes that degrade HA (Jiang et al., 2007).

Enzymatic Degradation: The enzymatic degradation of HA can be mediated by two major enzyme families: hyaluronidases and reactive oxygen species (ROS)-generating enzymes, as below.

Hyaluronidases: Hyaluronidases are enzymes that can specifically cleave the bonds within the HA polymer, resulting in the production of smaller HA fragments. There are several hyaluronidases, including HYAL1, HYAL2, and HYAL3, which exhibit different tissue expression patterns and substrate specificities (FIG. 13). These enzymes hydrolyze HA into smaller fragments of varying sizes (Jiang et al., 2007).

Reactive Oxygen Species (ROS)-Generating Enzymes: In addition to hyaluronidases, certain enzymes, such as NADPH oxidases and myeloperoxidases, can generate ROS as a byproduct of their activity. ROS can induce oxidative stress, leading to the breakdown of HA chains (Lassegue et al., 2012).

Fragmentation: Once HA is cleaved by hyaluronidases or subjected to oxidative stress, it can result in the generation of fragmented HA molecules of various sizes (FIG. 13). The size of the HA fragments can influence their biological activities and interactions with HA receptors (Jiang et al., 2007).

Clearance and Metabolism: The clearance and metabolism of HA fragments can be facilitated by several mechanisms. These include receptor-mediated endocytosis, which involves the internalization of HA fragments by specific receptors (e.g., CD44) on cell surfaces. The internalized HA fragments can be further metabolized by lysosomal enzymes, leading to their degradation into smaller components (Jiang et al., 2007).

Biological Effects: The HA fragments generated during degradation can exert various biological effects. Depending on their size, HA fragments can interact with different receptors, such as CD44 and Toll-like receptors (TLRs), triggering cellular responses related to inflammation, immune modulation, and tissue remodeling. The biological effects of HA fragments are diverse and depend on the specific context and receptors involved (Jiang et al., 2007).

Hyaluronan Signaling: Hyaluronan (HA) can influence cell behavior through binding cell surface receptors. High Molecular Weight (HMW) HA, which can reach up to 2×10\hat{ }4 kDa depending on the tissue type (Laurent and Fraser, 1992), can be regarded as mediating the homeostatic functions of HA, including tissue hydration, lubrication, and providing a support matrix for cells (Stem et al., 2006).

HMW HA, abundant in healthy tissues such as the vitreous humor of the eye and the synovial fluid of joints, acts as a lubricant and shock absorber. It also has anti-inflammatory and immunosuppressive effects. The immunosuppressive effect of HMW HA is suggested to prevent the accessibility of ligands to cell surface receptors (Stem et al., 2006a). In vitro studies have shown that HMW HA inhibits the expression of inflammatory chemokines (Jiang et al., 2005), and administration of aerosolized HMW HA (400 to 4000 kDa) has been shown to prevent exercise-induced bronchoconstriction in the lungs of asthmatic patients (Petrigni and Allegra, 2006).

However, following its degradation, the signaling properties of HA are altered. Low Molecular Weight-HA is generally considered to be more “biologically active” than the native HMW-HA. Low Molecular Weight HA has been shown to decrease endothelial barrier function, stimulate angiogenesis, cell migration, and immune cell recruitment, and induce expression of a host of inflammatory mediators. HA fragments, or LMW HA molecules, are shown to accumulate at the site of inflammation and injury, and have been observed to increase the expression of inflammatory cytokines and chemokines (Jiang et al., 2005; Teder et al., 2002).

Intracellularly, HA plays roles in signaling and functions, such as cell proliferation and migration. These roles can be facilitated through its interaction with specific HA receptors present on the cell surface and within the cell. The cluster determinant 44 (CD44) is a major HA receptor, impacting cell adhesion, migration, and activation of intracellular signaling cascades (Misra et al., 2015; Bourguignon et al., 2000). The lymphatic vessel endothelial HA receptor (LYVE-1) is involved in the turnover and degradation of HA by lymphatic endothelial cells (Jackson, 2004). The receptor for hyaluronate-mediated motility (RHAMM) interacts with HA both intracellularly and on the cell surface, regulating cell motility and proliferation (Turley et al., 2006). Additionally, the HA receptor for endocytosis (HARE) can be responsible for the clearance and degradation of HA, playing a vital role in intracellular HA signaling (Harris et al., 2020; Pandey & Weigel, 2014).

In situations of stress, HA can form unusually large molecules called stress cables. These HA cables arise intracellularly from more than one cell, forming a long cable-like structure connecting cells (Hascall et al., 2004). Studies have shown that HA cables are synthesized in several cell types under stress (Majors et al., 2003; Wang and Hascall, 2004). In human intestinal smooth muscle cell cultures, HA cable-like structures were synthesized after viral infection or endoplasmic reticulum stress induced by tunicamycin (de la Motte, 1999). HA cables were also detected in the glomeruli of kidney sections during hyperglycemia (Ren et al., 2009).

### The Differential Roles of HMW and LMW Forms in Inflammation:

As previously discussed, in its intact form, HMW-HA plays significant roles in tissue homeostasis, lubrication, and cell signaling (Jiang et al., 2007). However, under pathological conditions, such as tissue injury or inflammation, degradation of HA can lead to formation of HA fragments (Jiang et al., 2007). These fragments have been implicated in the induction and perpetuation of inflammation, contributing to the progression of several chronic diseases such as pulmonary fibrosis (Jiang et al., 2007), liver fibrosis (Stem, 2008), as well as rheumatoid arthritis (Weigel et al., 1997). In these conditions, the enzymatic degradation of HA by hyaluronidases and ROS-generating enzymes can result in the generation of fragmented HA molecules of varying sizes (Jiang et al., 2007; FIG. 13).

The size of the HA fragments can influence their biological activities and interactions with HA receptors (Jiang et al., 2007). For instance, in rheumatoid arthritis, elevated levels of HA fragments in the synovial fluid and blood have been associated with synovial inflammation and joint destruction. Furthermore, serum HA is seen elevated in patients with rheumatoid arthritis and other inflammatory arthropathies such as scleroderma and psoriatic arthritis (Engström-Laurent, 1987; Engström-Laurent, 1989). The clearance and metabolism of HA fragments are facilitated by receptor-mediated endocytosis, which involves the internalization of HA fragments by specific receptors (e.g., CD44) on cell surfaces (Jiang et al., 2007). Under pathological conditions, the balance between HA synthesis and degradation can be disrupted, leading to an accumulation of HA fragments of various size and perpetuating the inflammatory response. These fragments have been implicated in the induction and perpetuation of inflammation, contributing to the progression of several chronic diseases, as below:

Tissue Fibrosis in Chronic Inflammation: In various chronic inflammatory conditions where HA fragments are implicated, such as rheumatoid arthritis or chronic liver disease, persistent inflammation can lead to fibrogenesis. The prolonged inflammatory response can stimulate the activation of fibroblasts, which then produce excessive extracellular matrix components, including collagen, leading to tissue fibrosis.

Fibrosis in Lung Injury: HA fragments can contribute to lung injury and subsequent fibrosis. Inflammatory lung conditions, such as acute respiratory distress syndrome (ARDS) or idiopathic pulmonary fibrosis (IPF), can involve the release of HA fragments. These fragments can activate immune cells and promote the recruitment of fibroblasts, leading to excessive collagen deposition and fibrogenesis.

Liver Fibrosis: In chronic liver diseases, including hepatitis, alcohol-induced liver injury, or non-alcoholic steatohepatitis (NASH), HA fragments have been implicated in the activation of hepatic stellate cells (HSCs), which are key contributors to liver fibrosis. The activation of HSCs by HA fragments promotes their transformation into myofibroblasts, leading to increased collagen production and deposition in the liver. Therefore, the balance between HMW HA and LMW HA is crucial for maintaining tissue homeostasis. Changes in this balance, such as an increase in LMW HA due to tissue injury or inflammation, can contribute to disease progression. Alterations in these processes can contribute to disease progression, making them potential targets for therapeutic intervention in inflammatory joint diseases and other conditions.

High Molecular Weight (HMW) Hyaluronan: HMW HA, typically larger than 1,000 kDa, is abundant in healthy tissues, including the synovial fluid of joints. It can contribute to tissue integrity and provides a hydrated, anti-adhesive environment that facilitates cell movement. HMW HA has anti-inflammatory properties. It can inhibit the gathering of immune cells and suppresses the production of pro-inflammatory cytokines such as interleukin-1 (IL-1), interleukin-6 (IL-6), and tumor necrosis factor-alpha (TNF-alpha). It can suppress the expression of adhesion molecules like vascular cell adhesion molecule-1 (VCAM-1), which are involved in the recruitment of immune cells to the site of inflammation. Therefore, in the context of joint health, HMW HA helps maintain joint homeostasis and prevent inflammation.

Low Molecular Weight (LMW) Hyaluronan: On the contrary, LMW HA, which is usually less than 500 kDa, can be produced during tissue injury and inflammation. When tissues are injured or inflamed, HMW HA can be broken down into LMW HA by hyaluronidases and reactive oxygen species (ROS). Unlike HMW HA, LMW HA can have pro-inflammatory effects. It can stimulate the production of pro-inflammatory cytokines and promotes the recruitment of immune cells to the site of injury or inflammation. LMW HA can bind to toll-like receptors (TLRs), particularly TLR2 and TLR4, on the surface of immune cells such as macrophages and dendritic cells, triggering the release of pro-inflammatory cytokines. In the context of joints, an increase in LMW HA can contribute to joint inflammation and pain, as seen in conditions like osteoarthritis and rheumatoid arthritis.

Hyaluronan-Fragment Induced Inflammation: Muscle Stiffness and Joint Inflammation

In healthy synovial fluid, HMW-HA is the predominant form, contributing to the viscoelastic properties of the fluid and providing lubrication and shock absorption for the joint. However, in pathological conditions such as osteoarthritis (OA) and rheumatoid arthritis (RA), there is a shift towards LMW-HA (Greenwald and Moak, 1988). Serum HA in patients with rheumatoid arthritis rises during the first hour that patients are out of bed in the morning (Paimela, 1991). It has been shown that morning stiffness in these patients results from HA accumulation in the joints and muscles overnight, resulting in stiffness, and that stiffness improves as HA is mechanically driven out into the circulation by physical activity (Laurent, 1996).

Exercise in healthy individuals has been shown to increase serum HA significantly, which decreases rapidly to lower than resting levels by 30 minutes post exercise (Piehl-Aulin, 1985) indicating that the morning stiffness is due to overnight HA accumulation.

LMW HA has been found to induce the production of pro-inflammatory cytokines, such as IL-1, IL-6, and TNF-α, in synovial fibroblasts and other joint tissues. These cytokines contribute to the inflammation and joint damage seen in conditions like OA and RA (Campo et al., 2004). Additionally, LMW HA can stimulate the production of matrix metalloproteinases (MMPs), enzymes that break down the extracellular matrix and contribute to cartilage destruction in arthritis (Lisignoli et al., 2001). Furthermore, LMW HA can promote angiogenesis and the migration of immune cells to the joint, thereby exacerbating inflammation and joint damage (Campo et al., 2004). Clinical studies have shown that in OA patients, intraarticular injections of HMW HA, a treatment known as viscosupplementation, can relieve pain and improve joint function. This treatment can work, in part, by restoring the HA balance in the synovial fluid and suppressing the pro-inflammatory effects of LMW HA (Bagga et al., 2006). These findings highlight the roles of HMW and LMW HA in joint health and disease and indicate that modulating HA molecular weight could be a potential therapeutic strategy for inflammatory joint diseases.

### Hyaluronan-Fragment Induced Inflammation: Fibrogenesis

Fibrogenesis is the formation of fibrous tissue, often as a reaction or response to injury, inflammation, or disease. Low Molecular Weight-HA-fragments, typically generated during tissue injury and inflammation, have been shown to stimulate fibrogenesis (Jiang et al., 2005; Kessler et al., 2004). Furthermore, accumulating evidence indicates that HA fragment-induced inflammation can be associated with the activation of fibrogenesis, a process characterized by excessive extracellular matrix deposition and tissue fibrosis. HA fragment-induced inflammation has been linked to the activation of fibrogenesis in various tissues. Prolonged inflammation, triggered by HA fragments, can stimulate the activation of fibroblasts and other mesenchymal cells, leading to their differentiation into myofibroblasts. These activated myofibroblasts produce excessive extracellular matrix components, particularly collagen, leading to tissue fibrosis. These fragments can bind to Toll-like receptors (TLRs) on the surface of fibroblasts, the primary cells responsible for the production of extracellular matrix (ECM) proteins during fibrogenesis. Upon activation by LMW HA, fibroblasts can increase their production of ECM proteins such as collagen, leading to the development of fibrotic tissue (Liang et al., 2007). Toll-like receptors (TLRs) can play a significant role in the innate immune system by recognizing molecular patterns common to pathogens and initiating the production of cytokines for effective immune response. Research has highlighted the potential for Low Molecular Weight (LMW) Hyaluronan (HA) to activate certain TLRs, notably TLR2 and TLR4, triggering inflammation and fibrosis (Schaefer, L. 2014).

In addition, LMW HA can stimulate the production of transforming growth factor-beta (TGF-β), a potent pro-fibrotic cytokine, in various cell types. TGF-β can further enhance ECM production by fibroblasts and promote the differentiation of fibroblasts into myofibroblasts, a cell type with even higher ECM-producing capacity (Lee-Sayer et al., 2015). Notably, studies in animal models of liver and lung fibrosis have shown that HA fragments can promote the development of fibrotic tissue. Conversely, inhibiting the degradation of HA to LMW fragments, thereby preserving the High Molecular Weight (HMW) HA, has been shown to attenuate fibrogenesis in these models (Jiang et al., 2005).

### HA-Induced Systemic Inflammation:

HA fragments can trigger inflammatory responses through various signaling pathways. Toll-like receptors (TLRs), including TLR2 and TLR4, have been identified as key receptors mediating the recognition of HA fragments. Upon interaction with TLRs, HA fragments activate downstream signaling cascades, including the Toll-like receptor signaling pathway, leading to the activation of nuclear factor-kappa B (NF-κB) and mitogen-activated protein kinase (MAPK) pathways. These pathways can initiate the production of pro-inflammatory cytokines, such as interleukin-1β (IL-1β), tumor necrosis factor-α (TNF-α), and interleukin-6 (IL-6), further amplifying the inflammatory response.

Additionally, HA fragment-induced inflammation has been associated with the activation of the NOD-like receptor family pyrin domain-containing protein 3 (NLRP3) inflammation, leading to the release of pro-inflammatory cytokines IL-1β and IL-18. Furthermore, HA fragments can induce the generation of reactive oxygen species (ROS) and activate the complement system, both of which contribute to the inflammatory response. The interplay between these inflammatory pathways can create a positive feedback loop, promoting sustained inflammation and tissue damage.

### Model of Hyaluronan Degradation in Synovial Joints and Beyond:

A model for HA degradation is regulated by the uptake of HA by a cell surface receptor, CD44, followed by degradation by hyaluronidases and exoglycosidases. In their model, the authors indicate that HMW HA present at the cell surface is taken up by a cell surface receptor, CD44 (Culty et al., 1992), and is subsequently degraded by hyaluronidases and exoglycosidases. When stimulated, the hyaluronidases initiate the cleavage of HMW HA to generate HA fragments of 20 kDa. These fragments are transported to endosomes and then to lysosomes where they are further degraded by hyaluronidases into HA oligosaccharide units. Fragments are then released by exocytosis or further cleaved by the lysosomal enzymes into individual sugars (Stern, 2006).

The degradation of HA in the synovial fluid is similarly carried out by hyaluronidases (HYAL-1, HYAL-2, HYAL-3) that break down HMW-HA into smaller fragments. This can be relevant when analyzing the component of synovial fluid in rheumatoid arthritis patients. The synovial fluid of RA joints showed increased levels of HA but the ratio of LMW HA to that of HMW HA was increased (Cowman, 2015) and that disease severity is related to the presence of LMW HA.

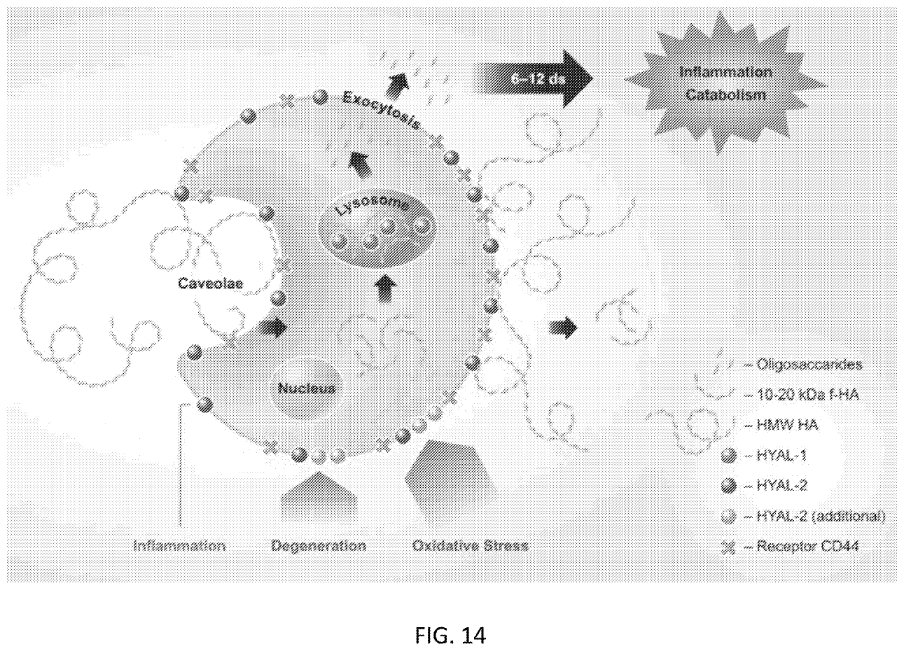

A similar mechanism of HA degradation has been described in the intervertebral disc (FIG. 14). These authors indicated that the process of HA cleavage is initiated by HYAL-2, a cell surface glycosylphospolipid anchored protein that acts in cooperation with CD44 (Bourguignon, 2004; Harda, 2007), by degrading HMW-HA into intermediate sized fragments of approximately 10-20 kDa (Girish, 2007). These fragments are partially taken up by the cell upon interaction with surface HA receptors (endocytosis) (Girish, 2007; Racine, 2012) and partially released into the extracellular space (Harada, 2007).

### REFERENCES

Hascall, V. C., Laurent, T. C., 1997. “Hyaluronan: structure and physical properties.” In: Laurent T. C. (eds) The Chemistry, Biology and Medical Applications of Hyaluronan and Its Derivatives. Wenner-Gren International Series, vol 70. Portland Press, London.
Scott J E and Heatley F. Biological properties of hyaluronan in aqueous solution are controlled and sequestered by reversible tertiary structures, defined by NMR spectroscopy. Biomacromolecules 2002; 3: 547-553.
Girish K S and Kemparaju K. The magic glue hyaluronan and its eraser hyaluronidase: abiological overview. Life Sci 2007; 80: 1921-1943.
Toole B P. Hyaluronan: from extracellular glue to pericellular cue. Nat Rev Cancer 2004; 4:528-539.
Fraser, J. R. E., Laurent, T. C., Laurent, U. B. G., 1997. “Hyaluronan: its nature, distribution, functions and turnover.” Journal of Internal Medicine, 242(1), pp. 27-33.
Prehm, P., 1984. “Synthesis of hyaluronate in differentiated teratocarcinoma cells. Mechanism of chain growth.” Biochem. J., 219, pp. 105-111.
DeAngelis, P. L., 1999. “Molecular directionality of polysaccharide polymerization by the Pasteurella multocida hyaluronan synthase.” J. Biol. Chem., 274, pp. 26557-26562.
Itano, N., Kimata, K., 2002. “Molecular cloning of human hyaluronan synthase.” Biochem. Biophys. Res. Commun., 222(3), pp. 816-820.
Volpi, N., Schiller, J., Stem, R., Soltés, L., 2009. “Role, metabolism, chemical modifications and applications of hyaluronan.” Current Medicinal Chemistry, 16(14), pp. 1718-1745.
Jiang D, Liang J, Noble P W. Hyaluronan in tissue injury and repair. Annu Rev Cell Dev Biol. 2007. Overview of hyaluronan's role in injury and disease.
Lassegue B., et al. “Reactive oxygen species (ROS)-generating oxidases in the normal and hypertensive cardiovascular system.” Circulation Research. 2012; 110(10):1362-1378.
Misra S, Hascall V C, Markwald R R, Ghatak S. (2015). Interactions between Hyaluronan and Its Receptors (CD44, RHAMM) Regulate the Activities of Inflammation and Cancer. Front Immunol.
Bourguignon L Y, Singleton P A, Zhu H, Zhou B. (2000). CD44 interaction with tiam1 promotes Rac1 signaling and hyaluronic acid-mediated breast tumor cell migration. J Biol Chem.
Jackson D G. (2004). Biology of the lymphatic marker LYVE-1 and applications in research into lymphatic trafficking and lymphangiogenesis. APMIS.
Turley E A, Noble P W, Bourguignon L Y. (2006). The hyaluronan receptor RHAMM: isoforms and intracellular signalling. Biochem Soc Symp.
Harris E N, Weigel J A, Weigel P H. (2020). The inner world of hyaluronan biotechnology. Nat Rev Rheumatol.
Pandey M S, Weigel P H. (2014). Hyaluronic acid receptor for endocytosis (HARE)-mediated endocytosis of hyaluronan, heparin, dermatan sulfate and acetylated LDL, but not chondroitin sulfate types A, C, D or E activates NF-κB regulated gene expression. J Biol Chem.
Engström-Laurent A, Hällgren R. Circulating hyaluronic acid levels vary with physical activity in healthy subjects and in rheumatoid arthritis patients. Relationship to synovitis mass and morning stiffness. Arthritis Rheum. 1987; 30(12):1333-1338.
Engström-Laurent A. Changes in hyaluronan concentration in tissues and body fluids in disease states. Ciba Found Symp. 1989; 143:233-240; discussion 240-7, 281-5.
Stem R. “Hyaluronan metabolism: A complex balancing act.” Journal of Investigative Dermatology. 2008; 128(6):1351-1354. DOI: 10.1038/sj.jid.5701213.
Weigel P. H., et al. “Increased hyaluronan in the airways of patients with cystic fibrosis.” American Journal of Respiratory Cell and Molecular Biology. 1997; 17(6):712-719.
Cowman M K, Lee H G, Schwertfeger K L, McCarthy J B, Turley E A. The Content and Size of Hyaluronan in Biological Fluids and Tissues. Front Immunol. 2015 Jun. 2; 6:261.
Schaefer, L. (2014). “Complexity of danger: the diverse nature of damage-associated molecular patterns.” Journal of Biological Chemistry, 289(51), 35237-35245.
Culty, M., Nguyen, H. A., Underhill, C. B., 1992. “The hyaluronan receptor (CD44) participates in the uptake and degradation of hyaluronan.” The Journal of cell biology, 116(4), pp. 1055-1062.
Girish K S, Kemparaju K (2007) The magic glue hyaluronan and its eraser hyaluronidase: a biological overview. Life Sci 80:1921-1943.
Racine R, Mummert M E (2012) Hyaluronan endocytosis: mechanisms of uptake and biological functions. Mol Regul Endocytosis 1:377-390.
Bourguignon L Y, Singleton P A, Diedrich F, Stem R, Gilad E (2004) CD44 interaction with Na+-H+ exchanger (NHE1) creates acidic microenvironments leading to hyaluronidase-2 and cathepsin B activation and breast tumor cell invasion. J Biol Chem 279:26991-27007.
Harada H, Takahashi M (2007) CD44-dependent intracellular and extracellular catabolism of hyaluronic acid by hyaluronidase-1 and -2. J Biol Chem 282:5597-5607.
Laurent, T. C., Fraser, J. R., 1992. “Hyaluronan: its nature, distribution, functions and turnover.” Journal of Internal Medicine, 242(1), pp. 27-33.
Stem, R., Asari, A. A., Sugahara, K. N., 2006. “Hyaluronan fragments: an information-rich system.” European Journal of Cell Biology, 85(8), pp. 699-715.
Jiang, D., Liang, J., Noble, P. W., 2005. “Hyaluronan in tissue injury and repair.” Annu. Rev. Cell Dev. Biol., 23, pp. 435-461.
Petrigni, G., Allegra, L., 2006. “Aerosolised hyaluronic acid prevents exercise-induced bronchoconstriction, suggesting novel hypotheses on the correction of matrix defects in asthma.” Pulm. Pharmacol. Ther., 19, pp. 166-171.
Hascall, V. C., Majors, A. K., De La Motte, C. A., Evanko, S. P., Wang. A., Drazba, J. A., Strong, S. A., Wight, T. N., 2004. “Intracellular hyaluronan: a new frontier for inflammation?” Biochim. Biophys. Acta., 1673, pp. 3-12.
Majors, A. K., Austin, R. C., de la Motte, C. A., Pyeritz, R. E., Hascall, V C., Kessler, S. P., Sen, G., Strong, S. A., 2003. “Endoplasmic reticulum stress induces hyaluronan deposition and leukocyte adhesion.” J. Biol. Chem., 278, pp. 47223-47231.
Wang, A., Hascall, V. C., 2004. “Hyaluronan structures synthesized by rat mesangial cells in response to hyperglycemia induce monocyte adhesion.” J. Biol. Chem., 279, pp. 10279-10285.
de la Motte, C. A., Hascall, V. C., Calabro, A., Yen-Lieberman, B., Strong, S. A., 1999. “Mononuclear leukocytes preferentially bind via CD44 to hyaluronan on human intestinal mucosal smooth muscle cells after virus infection or treatment with poly (I. C).” J. Biol. Chem., 274, pp. 30747-30755.
Ren, Y., Hascall, V. C., Wang, A., 2009. “Hyaluronan protects against lipopolysaccharide-stimulated nitric oxide production in macrophages by preventing RhoA activation and cytoskeletal rearrangement.” J. Biol. Chem., 284, pp. 13647-13654.
Teder, P., Vandivier, R. W., Jiang, D., Liang, J., Cohn, L., Pure, E., Henson, P. M., Noble, P. W., 2002. “Resolution of lung inflammation by CD44.” Science, 296, pp. 155-158.
Greenwald, R. A., & Moak, S. A. (1988). “Degradation of hyaluronic acid in synovial fluid by reactive oxygen species.” Free Radical Biology and Medicine, 5(3), 177-185.
Paimela L, Heiskanen A, Kurki P, Helve T, Leirisalo-Repo M. Serum hyaluronate level as a predictor of radiologic progression in early rheumatoid arthritis. Arthritis Rheum. 1991; 34(7):815-821.
Laurent T C, Laurent U B, Fraser J R. Serum hyaluronan as a disease marker. Ann Med. 1996; 28(3):241-253.
Piehl-Aulin K, Laurent C, Engström-Laurent A, Hellstrom S, Henriksson J. Hyaluronan in human skeletal muscle of lower extremity: concentration, distribution, and effect of exercise. J Appl Physiol (1985). 1991; 71(6):2493-2498.
Campo, G. M., Avenoso, A., Nastasi, G., Micali, A., Prestipino, V., Vaccaro, M., . . . & Campo, S. (2004). “Hyaluronan reduces inflammation in experimental arthritis by modulating TLR-2 and TLR-4 cartilage expression.” Biochimica et Biophysica Acta (BBA)—General Subjects, 1671(1), 36-45.
Lisignoli, G., Grassi, F., Zini, N., Toneguzzi, S., Piacentini, A., Guidolin, D., . . . & Facchini, A. (2001). “Anti-Fas-induced apoptosis in chondrocytes reduced by hyaluronan: evidence for CD44 and CD54 (intercellular adhesion molecule 1) involvement.” Arthritis & Rheumatism: Official Journal of the American College of Rheumatology, 44(8), 1800-1807.
Bagga, H., Burkhardt, D., Sambrook, P., & March, L. (2006). “Longterm effects of intraarticular hyaluronan on synovial fluid in osteoarthritis of the knee.” Journal of Rheumatology, 33(5), 946-950.
Jiang, D., Liang, J., Fan, J., Yu, S., Chen, S., Luo, Y., . . . & Noble, P. W. (2005). “Regulation of lung injury and repair by Toll-like receptors and hyaluronan.” Nature medicine, 11(11), 1173-1179.
Kessler, S., Rho, H. K., West, G. A., Fiocchi, C., Drazba, J. A., de la Motte, C. A. (2004). “Hyaluronan (HA) deposition precedes and promotes leukocyte recruitment in intestinal inflammation.” Clinical and translational science, 1(1), 57-61.
Liang, J., Jiang, D., Griffith, J., Yu, S., Fan, J., Zhao, X., . . . & Noble, P. W. (2007). “CD44 is a negative regulator of acute pulmonary inflammation and lipopolysaccharide-TLR signaling in mouse macrophages.” Journal of immunology (Baltimore, Md.: 1950), 178(4), 2469-2475.
Lee-Sayer, S. S. M., Dong, Y., Arif, A. A., Olsson, M., Brown, K. L., Johnson, P. (2015). “The where, when, how, and why of hyaluronan binding by immune cells.” Frontiers in immunology, 6, 150.
Yi Zhu, Ilja L. Kruglikov, Yucel Akgul, Philipp E. Scherer, Hyaluronan in adipogenesis, adipose tissue physiology and systemic metabolism, Matrix Biology, Volumes 78-79, 2019, Pages 284-291.

### Example 4

### Example 4: Method for Quantification and Characterization of Hyaluronan Fragments in Para-Facet Fluid and Muscle Tissue Samples

### Introduction

Disclosed are methods for can detecting and quantifying hyaluronan fragments in para-facet fluid and muscle tissue biopsy samples. In some embodiments, the patients are experiencing muscle stiffness. Quantifying total HA concentration and HA-fragment size distribution at various spinal segments can determine the source of extra-capsular synovial fluid leakage. This method can provide insights into the presence different HA fragment sizes in the para-facet fluid and muscle tissue and can provide accurate localization of a facet capsular failure with synovial fluid leakage. The methods involve collection of para-facet fluid and muscle tissue biopsy samples, extraction and/or purification of HA and HA-fragments from these samples, and detection, quantification and/or size characterization of hyaluronan fragments. The methods can use enzyme-linked immunosorbent assay (ELISA), flow cytometry techniques and other methodologies.

Non-Limiting List of Assays to Detect HA fragments are below:

Enzyme-linked immunosorbent assay (ELISA): This assay is a common method for detecting and quantifying hyaluronan. There are companies that provide hyaluronic acid ELISA kits.

The Hyaluronan Binding Protein Assay: The Hyaluronan Binding Protein (HABP) assay is a sandwich enzyme-linked immunosorbent assay (ELISA) used to measure the concentration of hyaluronan in a subject's serum or other biological samples.

Hyaluronan-Mediated Motility Receptor (HMMR) Binding Assay: This assay takes advantage of the binding specificity of the hyaluronan-mediated motility receptor (HMMR, also known as RHAMM) to hyaluronan. HMMR is a non-integral cell surface protein that interacts specifically with hyaluronan, aiding cell locomotion and contributing to wound repair, tissue remodeling, and other physiological processes. In the HMMR binding assay, typically, hyaluronan fragments are incubated with cells overexpressing HMMR (e.g., transfected cells). After washing away unbound hyaluronan, the amount of bound hyaluronan can be quantified, often by using specific antibodies against hyaluronan and appropriate detection methods (like fluorescence or chemiluminescence). This assay allows for the detection of hyaluronan and can provide information about its size distribution, as HMMR can have different affinities for hyaluronan of different sizes.

Hyaluronan-Mediated Motility Receptor (HMMR) Kits: Without wishing to be bound by theory, antibodies against HMMR can be used in flow cytometry, immunoprecipitation, or Western blot experiments.

Size Exclusion Chromatography or Electrophoresis: HA in a sample is separated by size using size-exclusion chromatography or gel electrophoresis. This can allow you to detect the presence and size distribution of HA fragments.

Mass Spectrometry: This technique can be used to accurately determine the size of HA fragments, particularly when combined with chromatographic methods to separate the fragments first.

Flow Cytometry Techniques: Flow cytometry is a technology that is used to analyze the physical and chemical characteristics of particles in a fluid as they pass by a beam of light. The use of flow cytometry in the detection of HA typically involves labeling of HA with a fluorescent probe, which can then be detected as cells pass through the flow cytometer. In some embodiments, the use of fluorescently tagged hyaluronan-binding protein (such as a tagged version of HMMR or another hyaluronan-binding protein like CD44), which binds to HA on the cell surface or in the extracellular matrix can be used. The cells are then analyzed using a flow cytometer, and the fluorescence signal intensity is measured for each cell, giving a measure of the amount of hyaluronan present. This technique allows for the rapid measurement of HA on a cell-by-cell basis, providing a high-throughput method for assessing the distribution of HA in a population of cells.

Immunohistochemistry: Detection of specific sizes of HA fragments can involve techniques such as gel electrophoresis and size exclusion chromatography, rather than antibody-based detection. This is because HA is a polysaccharide, and antibodies typically recognize and bind to proteins or peptides. However, there are some research studies that have developed antibodies that can recognize and bind to specific structures within HA, potentially allowing for the detection of certain HA fragments.

Imaging Technique to Detect HA: Several imaging techniques have been explored for non-invasive detection of polysaccharides, such as HA. In some embodiments, molecular probes that specifically target HA are used with a variety of imaging modalities. These probes typically involve molecules that either bind to HA directly or to its receptor CD44, and contain a label or tag that allows them to be visualized through imaging.

PET/SPECT Probes: PET (Positron Emission Tomography) and SPECT (Single-Photon Emission Computed Tomography) are nuclear medicine imaging techniques that can visualize physiological processes in the body. Probes for these techniques can contain radioisotopes such as carbon-11 or fluorine-18. These include probes like ¹¹C-labeled anti-CD44 antibodies or hyaluronic acid polymers that target the CD44 receptor, which is known to interact with HA and is often upregulated in disease. Other probes, like fluorine-18 labeled oligopeptides, contain hyaluronan affinity motifs that allow them to directly bind and quantify hyaluronan (van den Bosch et al., J Nucl Med, 2017).

Fluorescence Probes: These probes are designed for fluorescence imaging techniques and include molecules like near-infrared quantum dots or hyaluronan-binding peptides that are labeled with fluorescent dyes. These probes directly target and bind hyaluronan, allowing it to be visualized under fluorescence microscopy.

MRI Probes: Probes for Magnetic Resonance Imaging (MRI) can enhance contrast and allow more detailed visualization of specific molecules or structures. Gadolinium-labeled hyaluronic acid nanoparticles, for example, enhance contrast by shortening the T1 relaxation time in MRI. These probes can be used to visualize HA in tissues (Kheirollahi et al., Wiley Interdiscip Rev Nanomed Nanobiotechnol, 2020). Gadolinium-based contrast agents have been conjugated with HA-binding proteins to enable the visualization of HA in cartilage tissue.

OCT Probes: Optical Coherence Tomography (OCT) is an imaging technique that uses light to capture micrometer-resolution images from within optical scattering media (e.g., biological tissue). Probes for OCT, such as gold nanoparticles conjugated to hyaluronan-binding peptides, can enhance optical contrast and allow hyaluronan to be visualized (Li et al., Cancer Res, 2016).

Near-Infrared Fluorescence (NIRF) Imaging: This technique has been used to image HA in vivo by using fluorescently labeled HA-binding proteins or peptides. The fluorescence signal can be detected non-invasively using specialized imaging equipment.

Ultrasound Imaging: Ultrasound imaging with HA-targeted microbubbles can be used for detecting and quantifying HA in tissues. Microbubbles, used as gas-filled contrast agents in ultrasound imaging, can be coated with hyaluronan-binding ligands, enabling them to selectively bind to HA. In one study, microbubbles coated with hyaluronan binding protein (HABP) were used to image HA in arterial walls, providing a measure of vascular inflammation. Another study deployed microbubbles coated with a HA-binding peptide to detect liver fibrosis, successfully distinguishing between moderate and severe fibrosis models and controls (Correas, 2016; Yang, 2017).

### An Embodiment of a Method for Analyzing Para-Facet Fluid and Multifidus Muscle for HA is Described Below:

Sample Collection and Preparation: Collect para-facet fluid and multifidus muscle biopsy samples under sterile conditions. Handle biopsy samples carefully to avoid any degradation of the tissue. Immediately freeze samples in liquid nitrogen to preserve them, and then stored at −80° C. until further processing. Use a tissue homogenizer to break down the sample for the extraction of HA.
Hyaluronic Acid Extraction: Extract HA from the tissue samples using a tissue homogenizer and then perform an extraction (e.g., salt, mild detergent, and the like). Treat the supernatant with protease and nuclease to degrade proteins and nucleic acids, followed by a heat inactivation step.
HA Detection: Once the HA has been extracted, quantification of total HA concentration and/or distribution of HA fragments using one of the methods previously discussed, such as ELISA or HMMR Binding Assay can be used. Depending on the size of the HA fragments, different methods of separation can be required. Next, use the supernatant in an ELISA or HMMR Binding Assay according to the manufacturer's protocol. If using an ELISA, the supernatant can be added to a microplate that has been pre-coated with an antibody specific for HA. After a period of incubation to allow for binding, the plate can be washed to remove any unbound substances. A secondary antibody that binds to the HA can be added and a measurable signal (such as a color change) is produced. The signal can be measured using a suitable detector, such as a microplate reader.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method for diagnosing early stages of degenerative joint disease, comprising detecting synovial fluid or a component thereof outside of a synovial joint (extracapsular synovial fluid) in an animal.

2. The method of claim 1, wherein the animal comprises a human.

3. The method of any one of claim 1 or 2, wherein the synovial fluid or component outside of the synovial joint is detected in proximity to the synovial joint.

4. The method of any one of claims 1-3, wherein the synovial fluid or component outside of the synovial joint is detected in proximity to the synovial joint from which the synovial fluid originates.

5. The method of any one of claims 1-4, wherein the synovial joint is in a spine, elbow, thumb, foot, wrist, hip, shoulder or knee.

6. The method of any one of claims 1-5, wherein the synovial joint is in the spine, hip or knee.

7. The method of any one of claims 1-6, wherein the synovial joint is in the spine.

8. The method of any one of claims 1-7, wherein the synovial joint comprises a facet joint.

9. The method of any one of claims 1-8, wherein the facet joint comprises a cervical, thoracic or lumbar facet joint.

10. The method of claim 8, wherein the facet joint comprises the lumbar facet joint.

11. The method of any one of claims 1-10 wherein the synovial fluid or component outside of the synovial joint is from acute joint effusion.

12. The method of any one of claims 1-11, wherein the synovial fluid or component outside of the synovial joint leaks from the synovial joint.

13. The method of any one of claims 1-12, wherein the synovial fluid or component leaks from the synovial joint during physiologic gaping.

14. The method of any one of claims 1-13, wherein the synovial fluid or component leaks from the synovial joint during opening of the synovial joint during endpoints of flexion and/or extension.

15. The method of any one of claims 1-14, wherein the synovial joint is strained or injured.

16. The method of claim 15, wherein a synovial capsule of the synovial joint is injured.

17. The method of claim 15, wherein excessive axial load, rotational force, or a repetitive task leads to the strain or injury.

18. The method of any one of claims 2-17, wherein the human has a history of being sedentary.

19. The method of any one of claims 1-14, wherein the synovial joint is not inflamed.

20. The method of any one of claims 1-18, wherein the synovial joint, synovial capsule of the synovial joint, and/or synovium of the synovial joint is inflamed.

21. The method of claim 20, wherein the synovium is inflamed (synovitis).

22. The method of any one of claims 1-21, wherein the synovial fluid or component outside of the synovial joint originates from a tear or rupture of a synovial capsule.

23. The method of any one of claims 1-20, wherein the synovial fluid or component outside of the synovial joint does not originate from a tear or rupture of a synovial capsule.

24. The method of any one of claims 1-12, wherein the synovial fluid or component outside of the synovial Joint originates from a synovial cyst.

25. The method of claim 24, wherein the synovial fluid or component outside of the synovial joint originates from rupture of the synovial cyst.

26. The method of any one of claims 1-24, wherein the synovial fluid or component outside of the synovial joint does not originate from a synovial cyst.

27. The method of any one of claims 1-24, wherein the synovial fluid or component does not originate from rupture of a synovial cyst.

28. The method of any one of claims 1-27, wherein the synovial fluid or component outside of the synovial joint originates from leakage from a synovial joint that is proximal to the detected synovial fluid.

29. The method of any one of claims 1-28, wherein the synovial fluid or component outside of the synovial joint is detected in paraspinal musculature or in underlying ligaments flavum.

30. The method of any one of claims 1-28, wherein the synovial fluid or component outside of the synovial joint migrates into paraspinal musculature or underlying ligaments flavum.

31. The method of any one of claims 28-30, wherein the synovial fluid or component outside of the synovial joint inflames tissue surrounding the proximal synovial joint.

32. The method of claim 31, wherein the tissue surrounding the proximal synovial joint comprises, dorsally: ligament flavum, dural surface, epidural fat, paraspinal musculature and/or an exiting nerve root.

33. The method of claim 31 wherein the tissue surrounding the proximal synovial joint comprises, ventrally: multifidus muscle and/or a medial branch nerve.

34. The method of any one of claims 1-33, wherein the synovial fluid or component outside of the synovial joint does not originate from extracellular fluid.

35. The method of any one of claims 1-34, wherein the synovial fluid or component outside of the synovial joint does not originate from transcellular fluid in extracellular fluid.

36. The method of any one of claims 2-35, wherein the human has symptoms comprising acute back pain.

37. The method of any one of claims 2-36, wherein the human has symptoms comprising morning stiffness, paraspinal muscle spasms, posterior buttock pain, proximal leg pain.

38. The method of any one of claims 2-37, wherein the human does not have symptoms of chronic back pain.

39. The method of any one of claims 2-38, wherein the human does not have substantial radiographic presentation of lumbar degeneration (lumbar radiography is unremarkable).

40. The method of any one of claims 2-39, wherein the human has radiographic indication of facet joint scarring.

41. The method of any one of claims 2-40, wherein the human does not have fatty infiltration of the multifidus muscle.

42. The method of any one of claims 1-41, wherein the synovial fluid or component outside of the synovial joint is detected by imaging methods.

43. The method of any one of claims 1-42, wherein the synovial fluid or component outside of the synovial joint is detected by magnetic resonance imaging (MRI).

44. The method of any one of claims 1-42, wherein the synovial fluid or component outside of the synovial joint is detected by ultrasound.

45. The method of any one of claims 1-41, wherein the synovial fluid or component outside of the synovial joint is detected by an antibody specific for the component of synovial fluid.

46. The method of claim 45, wherein the component of synovial fluid comprises hyaluronic acid, proteoglycan 4, surface-active phosphor lipids, CXCL1, CXCL5, IL-6, IL-8, IL-10, TNFα, or a fragment thereof.

47. The method of claim 46, wherein the hyaluronic acid fragment comprises a low molecular weight (LMW) hyaluronic acid, an oligomer of hyaluronic acid, a monomer of hyaluronic acid, D-glucuronic acid, N-acetyl-D-glucosamine, or a combination thereof.

48. The method of any one of claims 45-47, wherein the antibody is attached to a detectable moiety.

49. The method of claim 48, wherein the detectable moiety comprises a radionuclide, fluorescent molecule, bioluminescent molecule, and/or light-emitting molecule.

50. The method of any one of claim 48 or 49, wherein the detectable moiety can be detected by imaging methods.

51. The method of claim 50, wherein the imaging method comprises PET (Positron Emission Tomography), SPECT (Single-Photon Emission Computed Tomography), fluorescence microscopy, Magnetic Resonance Imaging (MR), Optical Coherence Tomography (OCT), Near-Infrared Fluorescence (NIRF) Imaging, ultrasound imaging, or a combination thereof.

52. A method for identifying a human at risk for developing inflammation of tissue surrounding a facet joint, comprising detecting synovial fluid outside of, but in proximity to, the facet joint.

53. The method of claim 52, wherein the human does not have degenerative lumbar changes at the time the method is performed.

54. The method of any one of claim 52 or 53, wherein the human does not have a fatty infiltration in multifidus muscle at the time the method is performed.

55. The method of any one of claims 52-54, wherein the human does not have a synovial cyst associated with the facet joint at the time the method is performed.

56. The method of any one of claims 52-55, wherein the human does not have intervertebral disc disease at the time the method is performed.

57. The method of any one of claims 52-56, wherein the human presents with acute lumbar pain.

58. The method of claim 57, wherein the acute lumbar pain is recurrent.

59. The method of any one of claims 52-58, wherein the synovial fluid is detected using a substance that binds to a component of synovial fluid.

60. The method of claim 59, wherein the component of the synovial fluid comprises hyaluronic acid or a fragment thereof.

61. The method of claim 59, wherein the substance comprises an antibody, a hyaluronic acid-binding protein, or a combination thereof.

62. The method of claim 61, wherein the antibody is attached to a detectable moiety.

63. The method of claim 62, wherein the detectable moiety can be detected by imaging methods.

64. A method for detecting synovial fluid surrounding a synovial joint in a human patient with acute lumbar pain, comprising:
administering to the human patient a substance that can bind a component of extracapsular synovial fluid; and
detecting the substance bound to the component of extracapsular synovial fluid in the human patient.

65. The method of claim 64, wherein the component of synovial fluid comprises hyaluronic acid or a fragment thereof.

66. The method of claim 64, wherein the substance comprises an antibody or a hyaluronic acid-binding protein (HABP).

67. The method of claim 66, wherein the antibody or hyaluronic acid-binding protein is attached to a detectable moiety.

68. A method for identifying a human at risk for developing degenerative lumbar disease, comprising detecting extracapsular synovial fluid in proximity to a lumbar facet joint.

69. The method of claim 68, wherein the extracapsular synovial fluid is detected in ligament flavum, dural surface, epidural fat, paraspinal musculature, an exiting nerve root, multifidus muscle and/or a medial branch nerve.

70. The method of claim 68, wherein the synovial fluid comprises hyaluronic acid fragments.

71. The method of claim 70, wherein the hyaluronic acid fragments comprise low molecular weight (LMW) hyaluronic acid, a hyaluronic acid oligomer, a hyaluronic acid monomer, D-glucuronic acid, N-acetyl-D-glucosamine, or a combination thereof.

72. The method of claim 71, wherein the method detects the hyaluronic acid fragments.

73. A method for identifying a human at risk for developing degenerative lumbar disease, comprising detecting inflamed tissue surrounding a synovial joint.

74. The method of claim 73, wherein the tissue comprises ligament flavum, dural surface, epidural fat, paraspinal musculature, an exiting nerve root, multifidus muscle and/or a medial branch nerve.

75. The method of claim 73 or 74, wherein inflamed tissue is detected using a blood test.

76. The method of claim 75, wherein the blood test detects inflammatory markers.

77. A method for identifying a human at risk for developing degenerative lumbar disease, comprising detecting scarring, scabbing and/or plugs in a lumbar facet joint.

78. The method of claim 77, wherein the method comprises radiology.

79. A method for identifying a human at risk for developing degenerative lumbar disease, comprising detecting inflammatory molecules, or elevated levels thereof, in synovial fluid of a lumbar facet joint.

80. The method of claim 79, wherein the synovial fluid used in the method is obtained by arthrocentesis.

81. The method of claim 79, wherein the inflammatory molecules comprise interleukin-1 (IL-1), interleukin-6 (IL-6), tumor necrosis factor-alpha (TNF-alpha), low molecular weight (LMW) hyaluronic acid, transforming growth factor-beta (TGF-β), reactive oxygen species, or a combination thereof.

82. A method for identifying a human at risk for developing degenerative lumbar disease, comprising detecting inflammatory molecules, or elevated levels thereof, in a tissue sample obtained from tissue in proximity to a lumbar facet joint.

83. The method of claim 82, wherein the inflammatory molecules comprise interleukin-1 (IL-1), interleukin-6 (IL-6), tumor necrosis factor-alpha (TNF-alpha), low molecular weight (LMW) hyaluronic acid, transforming growth factor-beta (TGF-β), reactive oxygen species, or a combination thereof.

84. An early biomarker for degenerative lumbar disease in a human body, comprising a component of extracapsular synovial fluid.

85. The biomarker of claim 84, wherein the component of extra capsular synovial fluid is in complex with a substance that binds to the component of the extracapsular fluid.

86. The biomarker of claim 84 or 85, wherein the component of extracapsular synovial fluid comprises low molecular weight (LMW) hyaluronic acid or a fragment thereof.

87. The biomarker of claim 86 wherein the fragment thereof comprises a hyaluronic acid oligomer, a hyaluronic acid monomer, D-glucuronic acid, N-acetyl-D-glucosamine, or a combination thereof.

88. The biomarker of claim 85, wherein the substance comprises an antibody, a hyaluronic acid-binding protein, or a combination thereof.

89. The biomarker of claim 88, wherein the antibody is attached to a detectable moiety.

90. The biomarker of claim 89, wherein the detectable moiety comprises a radionuclide, fluorescent molecule, bioluminescent molecule, and/or light-emitting molecule.

91. A method of identifying a subject at risk for developing degenerative lumbar disease, comprising:
collecting a biopsy sample from the subject;
extracting hyaluronic acid or a fragment thereof from the biopsy sample;
detecting hyaluronic acid or a fragment thereof; and
identifying the subject as at risk or not at risk for developing degenerative lumbar disease.

92. The method of claim 91, wherein the biopsy sample comprises para-facet fluid, multifidus muscle, or a combination thereof.

93. The method of claim 91, wherein the hyaluronic acid or fragment thereof comprises low molecular weight (LMW) hyaluronic acid, a hyaluronic acid monomer, D-glucuronic acid, N-acetyl-D-glucosamine, or a combination thereof.

94. The method of claim 91, wherein extracting hyaluronic acid or a fragment thereof comprises:
homogenizing the biopsy sample;
extracting the homogenized sample with a buffer to generate a supernatant;
treating the supernatant with a protease, a nuclease, or a combination thereof; and
inactivating the protease, the nuclease, or a combination thereof with heat.

95. The method of claim 91, wherein detecting hyaluronic acid or a fragment thereof comprises performing a binding assay, size exclusion chromatography, electrophoresis, mass spectrometry, flow cytometry, immunochemistry, or an imaging technique.

96. The method of claim 95, wherein the binding assay comprises an enzyme-linked immunosorbent assay (ELISA), a hyaluronan binding protein assay, a hyaluronan-mediated motility receptor (HMMR) binding assay, or a combination thereof.

97. The method of claim 95, wherein the imaging technique comprises PET (Positron Emission Tomography), SPECT (Single-Photon Emission Computed Tomography), fluorescence microscopy, Magnetic Resonance Imaging (MRI), Optical Coherence Tomography (OCT), Near-Infrared Fluorescence (NIRF) Imaging, ultrasound imaging, or a combination thereof.
